(19) United States
(12) Patent Application Publication
(10) Pub. No.: US 2018/0092948 A1
(43) Pub. Date: Apr. 5, 2018

(54) PRODUCTION OF NITRO-FATTY ACIDS AND NITRO-HYDROCARBONS BY AMMONIA OXIDIZING BACTERIA

(71) Applicant: AOBIOME LLC, Cambridge, MA (US)

(72) Inventors: Larry Weiss, San Francisco, CA (US); David R. Whitlock, Cambridge, MA (US)

(21) Appl. No.: 15/563,122

(22) PCT Filed: Apr. 1, 2016
(86) PCT No.: PCT/US16/25553

§ 371 Date: Sep. 29, 2017

Related U.S. Application Data
(60) Provisional application No. 62/142,134, filed on Apr. 2, 2015.

(51) Int. Cl. A61K 35/74; A61K 9/00; A61K 31/04; A61K 31/201; A61K 31/202; A61P 29/00; A61K 45/06; A61P 17/10; A61P 17/06; A61P 17/00; A61P 13/12; A61P 9/00; A61P 11/00; A61P 25/00; A61P 5/00; C12P 13/00; C12Q 1/04

(52) U.S. Cl. CPC A61K 35/74; C12Q 1/04; A61K 31/04; A61K 31/201; A61K 31/202; A61P 29/00; A61K 45/06; A61P 17/10; A61P 17/06; A61P 17/00; A61P 13/12; A61P 9/00; A61P 11/00; A61P 25/00; A61P 5/00; C12P 13/008; A61K 9/0014

(57) ABSTRACT

This disclosure provides compositions containing ammonia oxidizing bacteria and methods of use, including treatment of disorders related to inflammation. Also provided are compositions containing nitro-fatty acids and nitro-hydrocarbons and methods of generating same using ammonia oxidizing bacteria.

### BACKGROUND

Ammonia oxidizing bacteria (AOB) are known to produce nitrite, nitric oxide, and nitrogen dioxide in the presence of sources of ammonia or urea. The reactive nitrogenous species react with unsaturated lipids, such as unsaturated fatty acids and unsaturated hydrocarbons, to form nitrated lipids, e.g., nitro-fatty acids and nitro-hydrocarbons.

Nitro-fatty acids and nitro-hydrocarbons have anti-inflammatory properties in vivo. Low levels of these compounds are produced endogenously and are involved in cell signaling events, including regulating inflammation, platelet function, and vessel relaxation. These compounds are labile and difficult to synthesize chemically. There is a need for sources of anti-inflammatory nitro-fatty acids and nitro-hydrocarbons that provide sufficient quantities to elicit therapeutic effects. There is also a need for methods to produce otherwise labile nitro-fatty acids and nitro-hydrocarbons for cosmetic and pharmaceutical use.

### SUMMARY

The invention is based in part on the discovery that AOB, when exposed to sources of ammonia or urea, such as on the skin of a mammal, produce nitro-fatty acids and nitro-hydrocarbons through the reaction of nitrogenous species produced by the AOB with unsaturated lipids normally found on the skin.

Accordingly, provided herein are compositions and methods comprising AOB and/or nitro-fatty acids and/or nitro-hydrocarbons, as well as their anti-inflammatory uses.

In one aspect, provided herein is a composition comprising ammonia oxidizing bacteria (AOB) and a nitro-hydrocarbon or a nitro-fatty acid.

The composition of claim 1, wherein the nitro-fatty acid or the nitro-hydrocarbon is present at a concentration of 0.0005 to 5% by weight (e.g., 0.0005 to 0.005%, 0.001 to 0.01%, 0.005 to 0.05%, 0.01 to 0.1%, 0.05 to 0.5%, 0.1 to 1%, or 0.5% to 5% by weight). In an embodiment, the nitro-fatty acid or the nitro-hydrocarbon is present at a concentration of 0.001 to 1% by weight.

In some embodiments, the composition comprises a concentration of nitro-fatty acid or nitro-hydrocarbon that is higher than that found on a skin of a mammal, e.g., human, e.g., that has not been administered an AOB. In some embodiments, the composition comprises a concentration of nitro-fatty acid or nitro-hydrocarbon that is higher than that found on a skin of a mammal, e.g., human, that has been topically administered an AOB (e.g., topically applied a composition comprising active AOB).

In some embodiments, the composition comprises a first nitro-fatty acid or nitro-hydrocarbon and a second nitro-fatty acid or nitro-hydrocarbon, where the first nitro-fatty acid or nitro-hydrocarbon is present at a concentration higher than that of the second nitro-fatty acid or nitro-hydrocarbon. In embodiments, the first nitro-fatty acid or nitro-hydrocarbon is present at a concentration at least 1.5-fold (e.g., at least 1.5-fold, 3-fold, 5-fold, 10-fold, 20-fold, 40-fold, 80-fold, 100-fold, 200-fold, 1000-fold, or higher) higher than the concentration of the second nitro-fatty acid or nitro-hydrocarbon.

In embodiments, the AOB comprise live AOB, dead AOB, or a mixture of live and dead AOB. In embodiments, 90% of fewer, e.g., 90%, 80%, 70%, 60%, 50%, 40%, 30%, 20%, 10%, 5%, 2.5%, 1%, or fewer, of the AOB are live. In other embodiments, 90% or fewer, e.g., 90%, 80%, 70%, 60%, 50%, 40%, 30%, 20%, 10%, 5%, 2.5%, 1%, or fewer, of the AOB are dead. In some embodiments, the composition comprises 90% of less, e.g., 90%, 80%, 70%, 60%, 50%, 40%, 30%, 20%, 10%, 5%, 2.5%, 1%, or less, by weight of AOB.

In some embodiments the ammonia oxidizing bacteria is selected from the group consisting of Nitrosomonas, Nitrosococcus, Nitrosospria, Nitrosocystis, Nitrosolobus, Nitrosovibrio, and combinations thereof. In an embodiment, the AOB is a D23 Nitrosomonas eutropha strain designated AOB D23-100 (deposited with the American Tissue Culture Collection (ATCC) on Apr. 8, 2014 having accession number PTA-121157).

In some embodiments, the nitro-fatty acid a nitroalkene fatty acid derivative or a nitrohydroxy fatty acid derivative. For example, the nitro-fatty acid comprises nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, or a nitrohydroxy derivative of linolenate, or a nitro derivative of sebaleic acid, or a nitro derivative of sapienic acid.

In embodiments, the nitro-hydrocarbon comprises nitro-squalene.

In embodiments, the composition is suitable for cosmetic use or pharmaceutical use.

For example, the composition is adapted for use as a cosmetic product. In an embodiment, the composition is in the form of a powder, cream, stick, aerosol, salve, or wipe. In an embodiment, the composition is disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, the composition comprises ammonia oxidizing bacteria at about 10¹⁰ to about 10¹³ colony forming units/Liter (CFU/L). In some embodiments, the composition comprises ammonia oxidizing bacteria at an amount between about 0.1 milligrams (mg) and about 1000 mg of ammonia oxidizing bacteria. In embodiments, the composition further comprises an excipient, e.g., an excipient described herein. In embodiments, the mass ratio of ammonia oxidizing bacteria to a pharmaceutically acceptable excipient or to a cosmetically acceptable excipient may be in a range of about 0.1 grams per liter to about 1 gram per liter.

In a second aspect, provided herein is a method of treating or preventing a disorder in a subject comprising topically administering to the subject a composition comprising AOB.

In embodiments, the method comprises topically administering to the subject a composition described herein.

In embodiments, the method comprises a step of selecting the subject based on (i) the need for an anti-inflammatory agent, (ii) the risk for developing an inflammatory disorder, or (iii) a medical history comprising a previous inflammatory disorder.

In embodiments, the subject (i) is in need of an anti-inflammatory agent, (ii) is at risk for developing an inflammatory disorder, or (iii) has previously had an inflammatory disorder.

In embodiments, the subject has previously been administered an anti-inflammatory agent, e.g., an anti-inflammatory agent other than a composition comprising AOB.

In some cases, the subject exhibited a partial response or no response to the previously administered anti-inflammatory agent.

In some cases, the method comprises administering to the subject an anti-inflammatory agent in combination with the composition comprising AOB.

In some examples, the anti-inflammatory agent is administered concurrently with, before, or after administration of the composition comprising AOB.

In embodiments, the anti-inflammatory agent and the composition comprising AOB are formulated together or separately.

In some embodiments, the method comprises administering a dose of the anti-inflammatory agent that is lower than an average recommended dose, e.g., an average recommended dose for a particular indication. In embodiments, combining an AOB with an anti-inflammatory agent decreases the dose normally required of the anti-inflammatory agent to achieve an anti-inflammatory effect, e.g., reduce a symptom of an inflammatory disorder.

In some embodiments, the method does not comprise administering an anti-inflammatory agent concurrently with, less than 6 months before (e.g., less than 6, 5, 4, 3, 2, or 1 month before), or less than 6 months after (e.g., less than 6, 5, 4, 3, 2, or 1 month after) administration of the composition comprising AOB. In embodiments, administration of the composition comprising AOB as a monotherapy, e.g., without co-administration with a separate anti-inflammatory agent, is effective to achieve an anti-inflammatory effect, e.g., reduce a symptom of an inflammatory disorder.

In some cases, the composition comprising AOB is administered, e.g., topically, at a dose of 3×10⁶ to 3×10⁷ AOB cells/cm² of skin.

In embodiments, the composition comprising AOB is administered every 1, 2, 3, 4, 5, 6, or 7 days, or every 1, 2, 3, 4, 5, 6, 7, 8, 9, or 10 weeks, or every month. In embodiments, the composition comprising AOB is administered for at least 1 week, e.g., at least 1, 2, 3, 4, 5, 6, 7, 8, 9, 10 weeks, or at least 1 month, e.g., at least 1, 2, 3, 4, 5, 6, 7, 8, 9, 10, 11, 12 months, or at least 1 year, e.g., at least 1, 2, 3, 4, 5, 6, 7, 8, 9, 10, or 15 years.

In some cases, the composition comprising AOB is formulated with an excipient, e.g., an excipient described herein, e.g., a pharmaceutically acceptable excipient or a cosmetically acceptable excipient. In some embodiments, the excipient is be suitable for one of topical, nasal, and pulmonary administration. In embodiments, the excipient is a surfactant. In some embodiments, the surfactant is selected from the group consisting of cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof.

In some cases, the composition comprising AOB further comprises a nitro-fatty acid or a nitro-hydrocarbon, e.g., as described herein.

In some embodiments, the disorder comprises a skin disorder, e.g., skin inflammation, acne, eczema, psoriasis, or rosacea. In some embodiments, the disorder is a renal, cardiovascular, pulmonary, neurological, or endocrine disorder.

In embodiments, the composition is administered to the surface of a body, e.g., to a portion of skin. The portion may be a facial area. The portion may be a lip. The portion may be an underarm.

In embodiments, the composition comprises an activated AOB, e.g., AOB in a growth state. In other embodiments, the composition comprises an AOB in an inactive state, e.g., a storage state. In some embodiments, the composition comprises a first compartment comprising the AOB in a storage state and a second compartment comprising an activator, e.g., ammonia or oxygen, where the first compartment and the second compartment are physically separated by a barrier. In some embodiments, the method comprises disrupting the barrier between the first compartment and the second compartment, where upon disruption of the barrier, the AOB and the activator mix or contact one another, thereby producing activated AOB. In embodiments, the method comprises depositing the composition comprising AOB on a skin surface of the subject prior to disruption of the barrier. In embodiments, the method comprises depositing the composition comprising AOB on a skin surface of the subject after disruption of the barrier.

In a third aspect, also provided herein is a method of manufacturing a nitro-fatty acid or nitro-hydrocarbon comprising incubating an AOB with an unsaturated fatty acid or unsaturated hydrocarbon.

In some embodiments, a mixture of AOB and unsaturated fatty acid/unsaturated hydrocarbon contains a concentration of 10̂7 to 10̂12 AOB cells/mL (e.g., 10̂7 to 10̂8, 10̂8 to 10̂9, 10̂9 to 10̂10, 10̂10 to 10̂11, or 10̂11 to 10̂12 AOB cells/mL), and a concentration of 0.001 to 1 millimoles/L (e.g., 0.001-0.01, 0.005-0.05, 0.01-0.1, 0.05-0.5, or 0.1-1 millimoles/L) of unsaturated fatty acid/unsaturated hydrocarbon.

In some embodiments, the method comprises separating the nitro-fatty acid or nitro-hydrocarbon from unreacted fatty acid/hydrocarbon and/or AOB, thereby manufacturing a purified nitro-fatty acid or nitro-hydrocarbon composition or preparation.

In certain embodiments, the separating step comprises filtration, centrifugation, chromatography (e.g., column chromatography), and/or solvent extraction.

In embodiments, the unsaturated fatty acid comprises oleic acid, linoleic acid, or arachidonic acid, sebaleic acid, or sapienic acid. In embodiments, the unsaturated hydrocarbon comprises squalene.

In embodiments, the purified nitro-fatty acid or nitro-hydrocarbon preparation comprises 50% or less, e.g., 50%, 40%, 30%, 20%, 10%, 5%, 2.5%, 1% or less, by weight of AOB, e.g., live or dead AOB. In embodiments, the purified nitro-fatty acid or nitro-hydrocarbon preparation comprises less un-nitrated unsaturated fatty acid or hydrocarbon than nitro-fatty acid or nitro-hydrocarbon. In embodiments, the purified nitro-fatty acid or nitro-hydrocarbon preparation comprises 50% or less, e.g., 50%, 40%, 30%, 20%, 10%, 5%, 2.5%, 1%, 0.5%, 0.1%, 0.05%, 0.01%, 0.005%, 0.001%, 0.0005%, 0.0001%, or less, by weight of the un-nitrated unsaturated fatty acid or hydrocarbon. In embodiments, the purified nitro-fatty acid or nitro-hydrocarbon preparation comprises 0.0001% or more, e.g., 0.0001%, 0.0005%, 0.001%, 0.005%, 0.01%, 0.05%, 0.1%, 0.5%, 1%, 5%, 10%, 20%, 30%, 40%, 50%, 60%, 70%, 80%, 90%, 95%, 99% or more of the nitro-fatty acid or nitro-hydrocarbon by weight.

In embodiments, the nitro-fatty acid or nitro-hydrocarbon comprises a nitroalkene fatty acid derivative or a nitrohydroxy fatty acid derivative. For example, the nitro-fatty acid comprises nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

In another aspect, also provided is a method of identifying an active strain of AOB, comprising detecting the level of nitro-fatty acid or nitro-hydrocarbon produced by a candidate AOB strain, wherein production of a level of a nitro-fatty acid or nitro-hydrocarbon above a control level indicates that the strain is active.

The disclosure contemplates all combinations of any one or more of the foregoing aspects and/or embodiments, as well as combinations with any one or more of the embodiments set forth in the detailed description and examples.

### DETAILED DESCRIPTION

Provided herein are compositions comprising nitro-fatty acids or nitro-hydrocarbons, e.g., produced by ammonia oxidizing bacteria. Also provided herein are compositions comprising ammonia oxidizing bacteria (AOB) in combination with a nitro-fatty acid or nitro-hydrocarbon. Also provided are methods of using AOB to treat or prevent a disorder, such as an inflammatory disorder. The present disclosure provides for methods of manufacturing nitro-fatty acids and nitro-hydrocarbons using AOB. The present disclosure also provides for methods of identifying active strains of AOB capable of generating nitro-fatty acids and nitro-hydrocarbons. Preparations, compositions, and formulations including, e.g., cosmetics and pharmaceutical compositions, comprising AOB are provided.

Ammonia-oxidizing bacteria (AOB) of the genus Nitrosomonas are Gram-negative obligate autotrophic bacteria with a unique capacity to generate nitrite and nitric oxide exclusively from ammonia as an energy source. They are widely present both in soil and water environments and are essential components of environmental nitrification processes. Due to the roles of nitrite and nitric oxide on human skin as important components of several physiological functions, such as vasodilation, skin inflammation and wound healing, these bacteria may have beneficial properties for both healthy and immunopathological skin conditions. These bacteria may be safe for use in humans because they are slow-growing, cannot grow on organic carbon sources, may be sensitive to soaps and antibiotics, and have never been associated with any disease or infection in animals or humans.

Ammonia oxidizing bacteria are ubiquitous Gram-negative obligate chemolithoautotrophic bacteria with a unique capacity to generate energy exclusively from the conversion of ammonia to nitrite.

In some embodiments, ammonia oxidizing bacteria catalyze the following reactions.

At a neutral pH, ammonia generated from ammonium around neutral pH conditions is the substrate of the initial reaction. The conversion of ammonia to nitrite takes place in two steps catalyzed respectively by ammonia monooxygenase (Amo) and hydroxylamine oxidoreductase (Hao), as follows:

NH₃+2H⁺+2e-+O₂→NH₂OH+H₂O (A)

NH₂OH+H₂O→NO₂⁻+4e-+5H⁺ (B)

In some instances, reaction B is reported as follows, to indicate nitrous acid (HNO₂) formation at low pH:

NH₂OH+H₂O→HNO₂+4e-+4H⁺

In certain embodiments, NH₄⁺ and NH₃ may be used interchangeably throughout the disclosure.

### Definitions

An ammonia oxidizing bacterium refers to a bacterium capable of oxidizing ammonia or ammonium to nitrite. This may be accomplished at a rate. The rate, e.g., a pre-determined rate, may refer to the conversion of ammonium ions (NH₄⁺) (e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute. Examples of ammonia oxidizing bacteria include Nitrosomonas eutropha strains, e.g., D23 and C91, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) on Apr. 8, 2014 having accession number PTA-121157.

As used herein, “axenic” refers to a composition comprising an organism that is substantially free of other organisms. For example, an axenic culture of ammonia oxidizing bacteria is a culture that is substantially free of organisms other than ammonia oxidizing bacteria. In some embodiments, “substantially free” denotes undetectable by a method used to detect other organisms, e.g., plating the culture and examining colony morphology, or PCR for a conserved gene such as 16S RNA. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic ammonia oxidizing bacteria.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, oxidation of ammonia to nitrite, oxidation of hydrogen sulfide, and oxidation of Fe2⁺ to Fe3⁺ Autotrophic bacteria of the present disclosure are incapable of causing infection.

To “culture” refers to a process of placing an amount of a desired bacterium under conditions that promote its growth, i.e., promoting cell division. The conditions can involve a specified culture medium, a set temperature range, and/or an agitation rate. Bacteria can be cultured in a liquid culture or on plates, e.g., agar plates.

“Activation,” as used herein, is used relative to autotrophic bacteria, e.g., ammonia oxidizing bacteria. Activation refers to any action that may place the ammonia oxidizing bacteria in a potentially more active state, e.g., a growth state. Activation may relate to stimulation of autotrophic bacteria, e.g., ammonia oxidizing bacteria, to assist in some way in the conversion of at least one of ammonia, ammonium ions, and urea into nitrite, nitric oxide, or nitric oxide precursors. Activation may relate to helping establish a bacterial colony, e.g., to allow for the autotrophic bacteria, e.g., ammonia oxidizing bacteria, to compete with other existing bacteria. Activation may relate to providing an environment that may favor sustainability and/or growth of autotrophic bacteria, e.g., ammonia oxidizing bacteria. Activation may relate to accelerating availability of the autotrophic bacteria, e.g., ammonia oxidizing bacteria to an environment or a surface. “Activation” may provide for ammonia oxidizing bacteria to be in an “activated” or “growth state.” “Activation” may take place with the use of an activator. The ammonia oxidizing bacteria may come into contact with the activator to provide an ammonia oxidizing bacteria in an “activated” or “growth” state. This may occur within or outside of a container, delivery device, or delivery system, e.g., within a first chamber, a second chamber, a mixing chamber, a third or additional chamber, or combinations thereof. The activator may be at least one of ammonia, ammonium ions, oxygen, or urea. The activator may be an ammonium salt, e.g., ammonium chloride or ammonium sulfate. The concentration of the activator, e.g., ammonium salt, e.g., ammonium chloride or ammonium sulfate may be in a range of about 10 micromolar to about 100 millimolar. In certain aspects the concentration of the activator, e.g., ammonium salt, e.g., ammonium chloride or ammonium sulfate may be in a range of about 0.5 mM to about 50 mM. The activator may be in a solution, suspension, a powder, e.g., crystalline form, a media, a buffer, or disposed in or provide as a suitable carrier for maintaining the activator. The ammonia oxidizing bacteria may be in any suitable form for maintaining the AOB in a desired state, e.g., a storage state, e.g., an aqueous suspension, gel, or powder form. The at least one of ammonia, ammonium ions, or urea may be in a medium or a buffer to promote growth of ammonia oxidizing bacteria, e.g., an AOB media or a growth media. A time-release, or controlled release urea may be used as an activator. Alternatively or in addition, in embodiments, activation can be accomplished by allowing oxygen to contact the AOB. In embodiments, activation of AOB comprises contacting AOB with oxygen. For example, if the concentration of O₂ is below the concentration of O₂ that AOB consumes, the AOB inactive. In embodiments, activation of AOB comprises contacting AOB with both O₂ and a source of ammonia or urea. In some embodiments, AOB is premixed with ammonia and is, e.g., O₂-free. In other embodiments, AOB is not premixed with ammonia (e.g., the AOB is ammonia-free) and is contacted with O₂. For example, AOB can be contacted with O₂ by placing AOB in an O₂ permeable container, e.g., a container comprising a polymer that has significant O₂ permeability. Without being bound by theory, in some embodiments, the metabolic activity of AOB during storage is low, so a low flux of O₂ is adequate to allow a sufficient O₂ level to build up in a container, e.g., a sufficient O₂ level to activate AOB.

“Actuation,” as used herein, means that some action is being taken, e.g., a process is being started or something is being put into motion. In some embodiments, actuation may refer to the breaking of a barrier of a container, or the initiation of movement of one or more contents of a container, e.g., delivery of one or more contents of the container to outside of the container, e.g., to a surface or an environment.

A “barrier,” as used herein, may mean any structure or configuration that may serve to obstruct passage or to maintain separation, e.g., between a first chamber and a second chamber of a container. The barrier may be in the form of a valve, e.g., a check valve, filtering material, film, wax, lipid, polymer, or controlled release material, e.g., slow release material. The barrier may be a material that upon actuation of a container, it may allow passage of contents from a first chamber into a second chamber, passage of contents from a second chamber into a first chamber, or both. The barrier may be disrupted upon actuation, e.g., through piercing, puncturing, stabbing, perforating, penetrating, splitting, opening or tearing the barrier. The barrier may be in a form of valve, e.g., a check valve, a flexible or inflexible material that may not degrade upon contact with one or more contents of the container, or a flexible or inflexible material that may degrade upon contact with one or more contents of the container, a filter material. The barrier may be made of any material suitable for its purpose, e.g., a material that may serve to obstruct passage or to maintain separation, e.g., a polymeric material or metal material.

In some embodiments, the states most relevant to the present disclosure are the state of growth, e.g., maximal growth, characterized by a pH of at least about 7.6, ammonia, trace minerals, oxygen and carbon dioxide. Another state may be characterized by a pH of about 7.4 or less and characterized by an absence of carbon dioxide. Under low carbon dioxide conditions, ammonia oxidizing bacteria, e.g., Nitrosomonas, continues to oxidize ammonia into nitrite and generates ATP, but lacking carbon dioxide, e.g., lacking sufficient carbon dioxide, to fix and generate protein, it instead generates polyphosphate, which it uses as an energy storage medium. This may allow the ammonia oxidizing bacteria to remain in a “storage state” for a period of time, e.g., a pre-determined period of time, for example, at least 1, 2, 3, 4, 5, 6, 7, days, 1, 2, 3, 4 weeks, 1, 2, 3, 4, 5, 6, 7, 8, 9, 10, 11, 12 months, 1, 2, 3, 4, or 5 years. In some embodiments, the ammonia oxidizing bacteria may remain in a storage state for at least about 6 months to about 1 year.

As used herein, “growth state” refers to autotrophic bacteria, e.g., ammonia oxidizing bacteria, in a state or in an environment, e.g., a media, e.g., a culture media, e.g., a growth media, that may have a pH of at least about 7.6. Levels of at least one of ammonia, ammonium ions, and urea may be between about 1 micromolar and 1000 millimolar. Levels of trace materials are between about 0.01 micromolar iron and 200 micromolar iron. Levels of oxygen are between about 5% and 100% oxygen saturation (e.g., of media). Levels of carbon dioxide are between about 20 ppm and 10% saturation (e.g., of media). In certain aspects, levels of at least one of ammonia, ammonium ions, and urea may be between about 10 micromolar and 100 millimolar. Levels of trace materials are between about 0.1 micromolar iron and 20 micromolar iron. Levels of oxygen are between about 5% and 100% oxygen saturation. Levels of carbon dioxide are between about 200 ppm and 5% saturation (e.g., of media).

As used herein, “polyphosphate loading state” refers to autotrophic bacteria, e.g., ammonia oxidizing bacteria, in a state or in an environment, e.g., a media, e.g., a culture media, e.g., a growth media, that may have a pH of about 7.4, or less. Levels of at least one of ammonia, ammonium ions, and urea are between about 1 micromolar and 2000 millimolar. Levels of trace materials are between 0.01 micromolar iron and 200 micromolar iron. Levels of oxygen are between about 0% and 100% O₂ saturation (e.g., of media). Levels of carbon dioxide are between/less than about zero and 400 ppm, and phosphate levels greater than about 1 micromolar. In certain aspects, levels of at least one of ammonia, ammonium ions, and urea are between about 10 micromolar and 200 millimolar. Levels of trace materials are between 0.1 micromolar iron and 20 micromolar iron. Levels of oxygen are between about 5% and 100% O₂ saturation. Levels of carbon dioxide are between/less than about zero and 200 ppm, and phosphate levels greater than about 10 micromolar.

The polyphosphate loading state may be induced for a period of time, e.g., a pre-determined period of time. The pre-determined period of time may the time period that allows sufficient polyphosphate accumulation in the ammonia oxidizing bacteria. This pre-determined period of time is the period of time suitable to provide for sufficient polyphosphate loading to allow for the ammonia oxidizing bacteria to be stored for an extended period of time. The pre-determined period of time may be at least partially based on a period of time of about 0.2-10 times, 0.3-5 times, 0.5-3 times, 0.5-1.5 times, or 0.5 to 1 times the doubling time for the ammonia oxidizing bacteria. The pre-determined period of time may be at least partially based on a period of time of about one doubling time for the ammonia oxidizing bacteria. In some embodiments, the pre-determined period of time is between about 8 hours and 12 hours. In some embodiments, the pre-determined period of time is about 10 hours. In some embodiments, the pre-determined period of time is about 24 hours.

A purpose of the polyphosphate loading state may be to provide AOB with sufficient ammonia, ammonium ions, and/or urea, and O₂ such that ATP can be produced, but to deny them CO₂ and carbonate such that they are unable to use that ATP to fix CO₂ and instead use that ATP to generate polyphosphate which may be stored by the bacteria.

As used herein, the term “storage state” refers to autotrophic bacteria, e.g., ammonia oxidizing bacteria, in a state or in an environment, e.g., a media, e.g., a culture media, e.g., a growth media, having a pH of about 7.4 or less (in some embodiments, the pH may be 7.6 or less). Levels of at least one of ammonia, ammonium ions, and urea are between about 1 and 1000 micromolar. Levels of trace materials are between about 0.1 and 100 micromolar. Levels of oxygen are between about 0 and 100% saturation (e.g., of media). Levels of carbon dioxide are between about 0 and 800 ppm. In certain aspects, levels of at least one of ammonia, ammonium ions, and urea are between about 10 and 100 micromolar. Levels of trace materials are between about 1 and 10 micromolar. Levels of oxygen are between about 0 and 100% saturation (e.g., of media). Levels of carbon dioxide are between about 0 and 400 ppm.

AOB are produced according to some embodiments of the present disclosure by generating AOB biomass during a growth state, then exposing the AOB to a polyphosphate loading state and then removing the media and resuspending the AOB in a buffer, e.g., a storage buffer (i.e., the storage state).

The ammonia oxidizing bacteria may remain in a “storage state” for a period of time, e.g., a pre-determined period of time, for example, at least 1, 2, 3, 4, 5, 6, 7, days, 1, 2, 3, 4 weeks, 1, 2, 3, 4, 5, 6, 7, 8, 9, 10, 11, 12 months, 1, 2, 3, 4, or 5 years. In some embodiments, the ammonia oxidizing bacteria may remain in a storage state for at least about 6 months to about 1 year. Upon revival, the viability of the ammonia oxidizing bacteria is at least about 50%, 60%, 70%, 80%, 90%, or 100% of the viability as of the ammonia oxidizing bacteria prior to storage e.g., in a growth state). In some embodiments, the preparation of ammonia oxidizing bacteria may be prepared, such that no more than 10%, 20%, 30%, 40%, 50%, 60%, or 70% of the ability to oxidize NH₄⁺ is lost upon storage at selected conditions.

The time that it takes to revive the ammonia oxidizing bacteria from a storage state (or a polyphosphate loading state) may be a pre-determined period of time. For example, the pre-determined period of time may be less than about 75 hours, or less than about 72 hours. The pre-determined period of time may at least partially based on a period time of about 0.2-10 times, 0.3-5 times, 0.5-3 times, 0.5-1.5 times, or 0.5 to 1 times the doubling time for the ammonia oxidizing bacteria. The pre-determined period of time may be at least partially based on a period of time of about one doubling time for the ammonia oxidizing bacteria. The pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be between about 5 minutes and 5 hours. The pre-determined period of time may be about 5-10 minutes, 10-15 minutes, 15-20 minutes, 20-25 minutes, 25-30 minutes, 30-45 minutes, 45-60 minutes, 60 minutes-1.5 hours, 1.5 hours-2 hours, 2 hours-2.5 hours, 2.5 hours-3 hours, 3 hours-3.5 hours, 3.5 hours-4 hours, 4 hours-4.5 hours, 4.5 hours-5 hours. In some embodiments, the pre-determined period of time may be about 2 hours. The pre-determined period of time, e.g., may be the time it may take to achieve revival of the ammonia oxidizing bacteria, e.g., achieve viability of the ammonia oxidizing bacteria as compared to the viability of the bacteria prior to storage (e.g., in a growth state), e.g., at least about 10%, 20%, 30%, 40%, 50%, 60%, 70%, 80%, 90%, 95%, 97%, 98%, 99%, or 100% viability

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery” or “consecutive delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered.

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

Natural products may be or comprise products for commercial purposes, and may refer to cosmetics, dietary supplements, and foods produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

As used herein, “presence” or “level” may refer to a qualitative or quantitative amount of a component, e.g., any one or more of an ammonia oxidizing bacteria, ammonia, ammonium ions, urea, nitrite, or nitric oxide. The presence or level may include a zero value or a lack of presence of a component.

The terms “polypeptide”, “peptide” and “protein” (if single chain) are used interchangeably herein to refer to amino acid polymers. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

As used herein, the term “surfactant”, includes compounds that may lower the surface tension, or interfacial tension, between two liquids or between a liquid and a solid. Surfactants may act as detergents, wetting agents, emulsifiers, foaming agents, and dispersants. Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K). and combinations thereof. Dr. Bronner's Castile soap and baby soap comprises water, organic coconut oil, potassium hydroxide, organic olive oil, organic fair deal hemp oil, organic jojoba oil, citric acid, and tocopherol.

As used herein, “transgenic” means comprising one or more exogenous portions of DNA. The exogenous DNA is derived from another organism, e.g., another bacterium, a bacteriophage, an animal, or a plant.

As used herein, “treatment of a disease or condition” refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein, “preventing” a disorder or condition refers to delaying the onset of a disorder or condition, e.g., by at least 1 day, e.g., at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, 5, 6, 7, 8, 9, 10 years or more. For example, preventing can refer to the delaying of a recurrence of a disorder or condition in a subject. Alternatively, preventing can refer to the delaying of a first occurrence of a disorder or condition in a subject.

As used herein a “therapeutically effective amount” refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” refers to the autotrophic bacteria's, e.g., ammonia oxidizing bacteria's, ability to oxidize ammonia, ammonium, or urea to nitrite at a pre-determined rate. In some embodiments, the rate refers to the conversion of ammonium ions (NH₄⁺) (e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute.

“Growth media” or “AOB media,” as referred to herein comprises the following components of Table 1 or Table 2:

TABLE 1

| | Weight/Volume | | Final Concentration | |
|---|---|---|---|---|
| | (in~1.5 L) | | (in~1.5 L) | |
| (NH₄)₂SO₄ (MW 132.14) | 4.95 | g | 50 | mM NH₄⁺ |
| KH₂PO₄ (MW 136.1) | 0.616 | g | 3.0 | mM |
| 1M MgSO₄ | 1.137 | ml | 0.76 | mM |
| 1M CaCl₂ | 0.3 | ml | 0.2 | mM |
| 30 mM FeCl₃/50 mM | 0.5 | ml | 10 μM/16.7 μM | |
| EDTA | | | | |
| 50 mM CuSO₄ | 30 | μl | 1.0 | μM |

Add 1400 ml ddH₂O to flask. Autoclave. Store at room temperature.
After autoclaving add:

| | | | |
|---|---|---|---|
| Phosphate Buffer | 100 | ml | 32 mM KH₂PO₄/ |
| | | | 2.7 mM NaH₂PO₄•H₂O |
| 5% Na₂CO₃ | 12 | ml | 0.04% |

TABLE 2

| | Batch medium | | Feeding solution | |
|---|---|---|---|---|
| | Weight/Volume | | Weight/Volume | |
| | (1 L) (Final | | (1 L) (Final | |
| | concentration) | | concentration) | |
| (NH₄)₂SO₄ (MW 132.14) | 3.3 | g | 13.2 | g |
| | (50 mM NH₄⁺) | | (200 mM NH₄⁺) | |
| KH₂PO₄ (MW 136.1) | 1.23 | g | 0.41 | g |
| | (9.0 | mM) | (3.0 | mM) |
| 1M MgSO₄ | 0.758 | ml | 0.758 | ml |
| | (0.76 | mM) | (0.76 | mM) |
| 1M CaCl₂ | 0.2 | ml | 0.2 | ml |
| | (0.2 | mM) | (0.2 | mM) |
| 30 mM FeCl₃/50 mM EDTA | 0.333 | ml | 0.333 | ml |
| | (10 μM/16.7 μM) | | (10 μM/16.7 μM) | |
| 50 mM CuSO₄ | 20 | μl | 20 | μl |
| | (1.0 | μM) | (1.0 | μM) |
| ddH₂O | 1000 | ml | 1000 | ml |

Autoclave each solution and store at room temperature.

### Nitrogenous Species, Nitro-Fatty Acids and Nitro-Hydrocarbons

Nitric oxide (NO) is a free radical that has a free, unbonded electron, which makes NO highly reactive with other free radicals. A common problem with poly-unsaturated fatty acids is their peroxidation via free-radical chain reactions while in contact with atmospheric oxygen.

Free-radical chain reactions proceed in a number of steps; as discussed in Huang et al. The chemistry behind antioxidant capacity assays. J Agric Food Chem. 2005 Mar. 23; 53(6):1841-56.

Initiation (via R₂N₂, or other endogenous free radical), propagation, termination and inhibition reactions involving free radicals are shown below.

### Initiation

R₂N₂→2R*+N₂ (1)

R*+O₂→ROO* (2)

ROO*+LH→ROOH+L* (3)

### Propagation

L*+O₂→LOO* (4)

LOO*+LH→LOOH+L* (5)

### Inhibition

LOO*+AH→LOOH+A* (6)

### Termination

A*+(n−1)LOO*→nonradical products (7)

LOO*+LOO*→nonradical products (8)

During the initiation step, a lipid free-radical is generated L*. During the propagation step, O₂ is added, forming a peroxidized lipid LOO* which abstracts a proton (H) from another lipid, generating a lipid free-radical L*, thereby propagating the chain reaction.

Free radicals react promiscuously with other molecules. As such, organisms have systems to trap and limit the concentrations of free-radicals to within physiological limits. Because free-radicals are highly reactive, small molecule free-radicals make ideal signaling molecules; the background is low, they are highly reactive, and can diffuse rapidly.

An advantage of NO as an antioxidant is that it is a small molecule, so diffusion is not sterically hindered. Also, it is lipid soluble gas, so it can penetrate hydrophobic membranes and arrive at hydrophobic regions of proteins and lipids.

Organisms evolved to utilize free-radicals as signaling molecules, including pathways that prevent damage from excess free-radicals. The physiology that responds to excess oxidative stress is the Phase I/Phase II response system. Phase I enzymes are expressed and generate electrophiles, which then trigger expression of Phase II systems that facilitate detoxification and excretion of those electrophiles.

When free-radicals react promiscuously in chain reactions, they can generate a large repertoire of electrophilic lipid radicals. However, many of these lipid radicals have lifetimes too short for isolation. See, e.g., Schopfer et al. Chem. Rev. 111.10 (2011):5997-6021. For example, when free radicals react with NO, a large repertoire of NO adducts is formed.

The skin is a rich source of lipids and hydrocarbons, with specialized sebaceous glands that produce mixtures of lipids, squalene, cholesterol, steroids, and lipid oxidation products. See, e.g., De Luca et al. Surface lipids as multifunctional mediators of skin responses to environmental stimuli. Mediators Inflamm. 2010; 2010:321494. Epub 2010 Oct. 20. NO can react with skin surface lipids to produce NO-lipid adducts. NO-lipid adducts (for example, nitro-hydrocarbons or nitro-fatty acids) have been shown to have anti-inflammatory properties and endogenous signaling effects. See, e.g., Khoo et al. Curr. Opin. Pharmacol. 10.2 (2010: 179-84; Freeman et al. J. Biol. Chem. 283.23 (2008):15515-19; Rudolph et al. Arterioscler. Thromb. Vasc. Biol. 30.5 (2010):938-45; Fazzari et al. PLoS One. 9.1 (2014):e84884; and Rubbo. Brazilian J. Med. Biol. Res. 46 (2013):728-34. For example, NO-lipid adducts play a role in activating the PPAR (Peroxisome proliferator-activated receptors). See, e.g., Kansanen et al. Activation of Stress Signaling Pathways by Electrophilic Oxidized and Nitrated Lipids. Free Radic. Biol. Med. 52.6 (2012):973-82; and Woodcock et al. J. Org. Chem. Biomimetic Nitration of Conjugated Linoleic Acid: Formation and characterization of Naturally Occurring Conjugated Nitrodienes. 79.1 (2014):25-33. Nitro-fatty acids have been shown to activate the Nrf2/Keap1 pathway, chaperone heat shock pathways, and to inhibit inflammatory responses (e.g., through inhibition of NF-kB). See, e.g., Vitturi et al. J. Biol. Chem. 288.35 (2013):25626-37.

The lipids and hydrocarbons produced by the skin, in addition to their oxidation and nitrated products, are reactive compounds, many of which have very short lifetimes. Thus, these compounds are often not stable enough to permit isolation, purification and identification. See, e.g., Guéraud et al. Chemistry and biochemistry of lipid peroxidation products. Free Radic Res. 2010 October; 44(10):1098-124; Rubbo et al. Nitric oxide inhibition of lipoxygenase-dependent liposome and low-density lipoprotein oxidation: termination of radical chain propagation reactions and formation of nitrogen-containing oxidized lipid derivatives. Arch Biochem Biophys. 1995 Dec. 1; 324(1):15-25.

Exemplary nitro-fatty acids include nitroalkene fatty acid derivatives and nitrohydroxy fatty acid derivatives. In some cases, a nitro-fatty acid comprises nitro-oleic acid (e.g., 9-nitro-octadecenoid acid or 10-nitro-octadecenoic acid), nitro-conjugated linoleic acid (e.g., 9-cis,11-trans-octadecadienoic acid, 12-nitro-octadeca-9,11-dienoic acid, 9-nitro-octadeca-9,12-dienoic acid, 10-nitro-octadeca-9,12-dienoic acid, 12-nitro-octadeca-9,12-dienoic acid, or 13-nitro-octadeca-9,12-dienoic acid), nitro-stearic acid (e.g., 9- and/or 10-nitro-octadecanoid acid), nitroarachidonic acid, a nitrohydroxy derivative of oleate, a nitrohydroxy derivative of linoleate, or a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid. In some embodiments, the nitro-hydrocarbon comprises nitro-squalene.

There is a need for methods of producing anti-inflammatory nitro-fatty acids and nitro-hydrocarbons that overcome their labile nature.

Compositions and methods described herein are based in part on the discovery that AOB produces NO, nitrogen dioxide, and nitrite in the presence of ammonia and/or urea. These reactive nitrogenous species react with unsaturated lipids, e.g., unsaturated fatty acids or hydrocarbons, many of which are found in/on the skin of mammals. Reaction of nitrogenous species with unsaturated fatty acids and unsaturated hydrocarbons (e.g., squalene and arachidonic acid derivatives) generates nitro-fatty acids and nitro-hydrocarbons, many of which have been shown to have anti-inflammatory properties, including at nanomolar concentrations.

Generating nitro-fatty acids and nitro-hydrocarbons in situ (e.g., on a skin of a subject) via natural means (e.g., by using AOB in situ), in accordance with some methods described herein, provides a number of advantages that are not limited by those listed herein. For example, by utilizing natural processes under physiological control (e.g, in situ generation of NO, nitrite and NOx from ammonia released by sweat glands), the production of nitro-fatty acids and nitro-hydrocarbons can be physiologically controlled (e.g, via the release of ammonia). In some embodiments, utilizing this natural control ensures that appropriate amounts of nitro-fatty acids and nitro-hydrocarbons are generated and that the nitro-fatty acids and nitro-hydrocarbons are generated in a location where they have been evolved to function as signaling compounds. Once generated, nitro-lipids cannot diffuse very far before reacting or signaling to downstream effector molecules, as they are labile with very short lifetimes. In some embodiments, the compositions and methods described herein advantageously generate nitro-lipids in situ, e.g., at the site of a skin disorder.

Achieving an appropriate dose of a highly reactive signaling molecule, such as a nitro-lipid, can be difficult. Doses that are too high can cause non-specific reactions and toxicity. Doses that are too low can be inefficacious for treating or preventing a disorder. When the dose is generated in situ, e.g., from endogenously generated precursors in combination with AOB, as described herein, the dose is regulated in part by physiology (e.g., the amount of endogenously generated precursors, e.g., ammonia, unsaturated fatty acids or hydrocarbons), and not by the amount of a final drug product applied. For example, organisms have regulatory pathways that reduce ammonia release when too much ammonia is generated. In some examples, when AOB is applied to a site on/in a subject, this regulatory mechanism limits the NO, nitrite, and/or NOx level that can be generated by AOB, e.g., limits the level of reactive nitrogenous species generated to a safe level.

In some embodiments, AOB generate NO and nitrite that are substantially free from non-physiological nitro-species, such as nitrosoamines, which can be toxic at non-physiological levels.

Applying AOB to the skin allows for the nitrogenous species produced by AOB to penetrate the skin and generate nitro-fatty acids and nitro-hydrocarbons. Topical application of AOB permits the in situ generation of anti-inflammatory nitro-fatty acids and nitro-hydrocarbons that would otherwise be too unstable to manufacture separately without the use of AOB. The AOB containing compositions generates higher levels of anti-inflammatory nitro-fatty acids and nitro-hydrocarbons in situ than would normally be present on the skin of a mammal, e.g., a human. These nitro-fatty acids and nitro-hydrocarbons can act on the skin surface or beneath the skin surface to counter inflammation. Alternatively, the nitro-fatty acids and nitro-hydrocarbons can diffuse into the bloodstream of a mammal to mediate anti-inflammatory effects systemically. Thus, the AOB-containing compositions are useful to treat or prevent disorders related to inflammation. The AOB-containing compositions can be in the form of cosmetics or pharmaceutical products.

AOBs are also useful for the manufacture of nitro-fatty acids and nitro-hydrocarbons in vitro.

### Ammonia Oxidizing Bacteria (AOBs)

Autotrophic ammonia oxidizing bacteria, which may be referred to herein as AOBs or AOB, are obligate autotrophic bacteria as noted by Alan B. Hooper and A. Krummel at al. Alan B. Hooper, Biochemical Basis of Obligate Autotrophy in Nitrosomonas europaea, Journal of Bacteriology, February 1969, p. 776-779. Antje Krummel et al., Effect of Organic Matter on Growth and Cell Yield of Ammonia-Oxidizing Bacteria, Arch Microbiol (1982) 133: 50-54. These bacteria derive all metabolic energy only from the oxidation of ammonia to nitrite with nitric oxide (NO) as an intermediate product in their respiration chain and derive virtually all carbon by fixing carbon dioxide. They are incapable of utilizing carbon sources other than a few simple molecules.

Ammonia oxidizing bacteria (AOB) are widely found in the environment, and in the presence of ammonia, oxygen and trace metals will fix carbon dioxide and proliferate. AOB may be slow growing and toxic levels of ammonia may kill fish and other organisms before AOB can proliferate and reduce ammonia to non-toxic levels. Slow growth of AOB also may delay the health benefits of the NO and nitrite the AOB produce when applied to the skin.

Supplementing the aquarium, skin, or process with sufficient viable AOB grown and stored for that purpose is desired. AOB do not form spores, so storage in the dry state with high viability is difficult, and storage in the wet state leaves them metabolically active.

Decay of nitrifying capacity during storage of AOB for wastewater treatment has been studied, as for example (Munz G, Lubello C, Oleszkiewicz J A. Modeling the decay of ammonium oxidizing bacteria. Water Res. 2011 January; 45(2): 557-64. Oi: 10.1016/j.watres.2010.09.022.)

Growth, prolonged storage, and restoration of activity of Nitrosomonas is discussed by Cassidy et al. (U.S. Pat. No. 5,314,542) where they disclose growing Nitrosomonas, removing toxic waste products, storing in sterile water of appropriate salinity for periods of time up to one year, and then reviving by adding buffer (CaCO₃) and 200 ppm, of ammonium, which reviving takes 72 hours.

The present disclosure provides that if AOB are kept under conditions of low carbon dioxide but with sufficient oxygen and ammonia, where they accumulate polyphosphate for a period of about one doubling time (˜10 hours), then they accumulate sufficient polyphosphate to greatly extends their storage viability, storage time and accelerate their revival both with and without addition of buffer and ammonia as disclosed by Cassidy et al.

As obligate autotrophs, AOB synthesize protein via the fixing of CO₂ using the energy and reducing equivalents generated by the oxidation of ammonia to nitrite. Growth requires ammonia, oxygen, minerals and carbon dioxide.

Nitrosomonas may exist in several metabolic states, according to “Polyphosphate and Orthophosphate Content of Nitrosomonas europaea as a Function of Growth” by K. R. Terry and A. B. Hooper, Journal of Bacteriology, July 1970, p. 199-206, Vol. 103, No. I.

The AOBs contemplated in this disclosure may comprise mutations relative to wild-type AOBs. These mutations may, e.g., occur spontaneously, be introduced by random mutagenesis, or be introduced by targeted mutagenesis. For instance, the AOBs may lack one or more genes or regulatory DNA sequences that wild-type AOBs typically comprise. The AOBs may also comprise point mutations, substitutions, insertions, deletions, and/or rearrangements relative to the sequenced strain or a wild-type strain. The AOBs may be a purified preparation of optimized AOBs.

In certain embodiments, the AOBs are transgenic. For instance, it may comprise one or more genes or regulatory DNA sequences that wild-type ammonia oxidizing bacteria lacks. More particularly, the ammonia oxidizing bacteria may comprise, for instance, a reporter gene, a selective marker, a gene encoding an enzyme, or a promoter (including an inducible or repressible promoter). In some embodiments the additional gene or regulatory DNA sequence is integrated into the bacterial chromosome; in some embodiments the additional gene or regulatory DNA sequence is situated on a plasmid.

In some embodiments, the AOBs differ by at least one nucleotide from naturally occurring bacteria. For instance, the AOBs may differ from naturally occurring bacteria in a gene or protein that is part of a relevant pathway, e.g., an ammonia metabolism pathway, a urea metabolism pathway, or a pathway for producing nitric oxide or nitric oxide precursors. More particularly, the AOBs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites® II in vitro Mutagenesis Systems (Promega Corp., Madison, Wis.); Erase-a-Base® System (Promega, Madison, Wis.); GeneTailor™ Site-Directed Mutagenesis System (Invitrogen, Inc., Carlsbad, Calif.); QuikChange® II Site-Directed Mutagenesis Kits (Stratagene, La Jolla, Calif.); and Transformer™ Site-Directed Mutagenesis Kit (BD-Clontech, Mountain View, Calif.).

In some embodiments of the disclosure, the ammonia oxidizing bacteria may be axenic. The preparation, e.g., formulation, e.g., composition, of ammonia oxidizing bacteria may comprise, consist essentially of, or consist of axenic ammonia oxidizing bacteria. The ammonia oxidizing bacteria may be from a genus selected from the group consisting of Nitrosomonas, Nitrosococcus, Nitrosospria, Nitrosocystis, Nitrosolobus, Nitrosovibrio, and combinations thereof.

In some embodiments, the preparation of ammonia oxidizing bacteria may comprise a concentration or amount of ammonia oxidizing bacteria in order to at least partially treat a condition or disease. The preparation of ammonia oxidizing bacteria may comprise a concentration or amount of ammonia oxidizing bacteria in order to alter, e.g., reduce or increase, an amount, concentration or proportion of a bacterium, or genus of bacteria, on a surface, e.g., a skin surface. The bacteria may be non-pathogenic or pathogenic, or potentially pathogenic. The preparation of AOB can comprise a concentration or amount of AOB in order to produce a detectable level of nitro-hydrocarbons or nitro-fatty acids on a surface, e.g., skin surface.

In some embodiments, the preparation of ammonia oxidizing bacteria may comprise between about 10⁸ to about 10¹⁴ CFU/L. The preparation may comprise at least 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/L. In some embodiments, the preparation may comprise at least 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/ml.

In certain aspects, the preparation may comprise between about 1×10⁹ CFU to about 10×10⁹ CFU. In certain aspects, the preparation may comprise between about 1×10⁹ CFU/L to about 10×10⁹ CFU/L.

In some embodiments, the preparation of ammonia oxidizing bacteria may comprise between about 0.1 milligrams (mg) and about 1000 mg of ammonia oxidizing bacteria. In certain aspects, the preparation may comprise between about 50 mg and about 1000 mg of ammonia oxidizing bacteria. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

In some embodiments, the preparation of ammonia oxidizing bacteria may comprise a mass ratio of ammonia oxidizing bacteria to an excipient, e.g., a pharmaceutically acceptable excipient or a cosmetically acceptable excipient in a range of about 0.1 grams per liter to about 1 gram per liter. The preparation may comprise a mass ratio of ammonia oxidizing bacteria to an excipient in a range of about 0.1-0.2, 0.2-0.3, 0.1-0.5, 0.2-0.7, 0.5-1.0, or 0.7-1.0 grams per liter.

In some embodiments, the preparation of ammonia oxidizing bacteria may be in a growth state. A growth state may be provided by exposing ammonia oxidizing bacteria to an environment that may promote growth. The growth state may be a state, e.g., ammonia oxidizing bacteria in an environment that allows immediate availability of ammonia oxidizing bacteria to convert ammonium ions (NH₄⁺) to nitrite (NO₂⁻). The growth state may comprise providing ammonia oxidizing bacteria in an environment having a pH of greater than about 7.6. The growth state may also comprise providing ammonia oxidizing bacteria in an environment having ammonia, ammonium ions, and/or urea, trace minerals and sufficient oxygen and carbon dioxide, as described above.

In some embodiments, the preparation of ammonia oxidizing bacteria may be in a polyphosphate loading state, wherein the state or the environment, e.g., a media, e.g., a culture media, e.g., a growth media, may have a pH of less than about 7.4. Levels of components are selected to provide AOB with ammonia and oxygen such that ATP can be produced, but to deny them carbon dioxide and carbonate such that they are unable to use that ATP to fix carbon dioxide and instead use that ATP to generate polyphosphate which may be stored.

In some embodiments, the preparation of ammonia oxidizing bacteria may be in a storage state. A storage state may be defined as ammonia oxidizing bacteria in an environment in which they may be stored to be later revived. The storage state may be a state, e.g., ammonia oxidizing bacteria in an environment that allows availability of ammonia oxidizing bacteria after being revived, e.g., after being place in an environment promoting a growth state for a pre-determined period of time.

The storage state may comprise providing ammonia oxidizing bacteria in an environment having a pH of less than about 7.4. The storage state may also comprise providing ammonia oxidizing bacteria in an environment having ammonia, ammonia ions, and/or urea, trace minerals, oxygen, and low concentrations of carbon dioxide, as described above.

Storage may also be accomplished by storing at 4° C. for up to several months. The storage buffer in some embodiments may comprise 50 mM Na₂HPO₄-2 mM MgCl₂ (pH 7.6).

In some embodiments, ammonia oxidizing bacteria may be cyropreserved. A 1.25 ml of ammonia oxidizing bacteria mid-log culture may be added to a 2 ml cryotube and 0.75 ml of sterile 80% glycerol. Tubes may be shaken gently, and incubate at room temperature for 15 min to enable uptake of the cryoprotective agents by the cells. The tubes may be directly stored in a −80° C. freezer for freezing and storage.

For resuscitation of cultures, frozen stocks may be thawed on ice for 10-20 minutes, and then centrifuged at 8,000×g for 3 minutes at 4° C. The pellet may be washed by suspending it in 2 ml AOB medium followed by another centrifugation at 8,000×g for 3 minutes at 4° C. to reduce potential toxicity of the cryoprotective agents. The pellet may be resuspended in 2 ml of AOB medium, inoculated into 50 ml of AOB medium containing 50 mM NH₄⁺, and incubated in dark at 30° C. by shaking at 200 rpm.

In some embodiments, the preparation of ammonia oxidizing bacteria may comprise ammonia oxidizing bacteria in a storage state and/or ammonia oxidizing bacteria in a polyphosphate loading state, and/or ammonia oxidizing bacteria in a growth state.

Without wishing to be bound by theory, by maintaining ammonia oxidizing bacteria under conditions or in an environment of low carbon dioxide, with sufficient oxygen and ammonia, they may accumulate polyphosphate for a pre-determined period, e.g., for a period of about one doubling time, e.g., for about 8-12 hours, e.g., for about 10 hours. The ammonia oxidizing bacteria may accumulate sufficient polyphosphate to extend their storage viability, storage time, and accelerate their revival. This may occur with or without the addition of buffer and ammonia.

The presence of sufficient stored polyphosphate may allow the ammonia oxidizing bacteria the ATP resources to maintain metabolic activity even in the absence of ammonia and oxygen, and to survive insults that would otherwise be fatal.

The process of oxidation of ammonia to generate ATP has two steps. The first step is the oxidation of ammonia to hydroxylamine by ammonia monoxoygenase (Amo), followed by the conversion of hydroxylamine to nitrite by hydroxylamine oxidoreductase (Hao). Electrons from the second step (conversion of hydroxylamine to nitrite) are used to power the first step (oxidation of ammonia to hydroxylamine).

If an ammonia oxidizing bacteria does not have hydroxylamine to generate electrons for Amo, then hydroxylamine is not available for Hao. For example, acetylene irreversibly inhibits the enzyme crucial for the first step in the oxidation of ammonia to nitrite, the oxidation of ammonia to hydroxylamine. Once AOB are exposed to acetylene, Amo is irreversibly inhibited and new enzyme must be synthesized before hydroxylamine can be generated. In a normal consortium biofilm habitat, AOB may share and receive hydroxylamine form other AOB (even different strains with different susceptibilities to inhibitors) and so the biofilm tends to be more resistant to inhibitors such as acetylene than an individual organism. AOB can use stored polyphosphate to synthesize new Amo, even in the absence of hydroxylamine.

Any embodiment, preparation, composition, or formulation of ammonia oxidizing bacteria discussed herein may comprise, consist essentially of, or consist of optionally axenic ammonia oxidizing bacteria.

### Methods of Producing Ammonia Oxidizing Bacteria

Methods of culturing various ammonia oxidizing bacteria, e.g., Nitrosomonas species are known in the art. Ammonia oxidizing bacteria may be cultured, for example, using media described in Table 1 or Table 2, above.

Ammonia oxidizing bacteria may be grown, for example, in a liquid culture or on plates. Suitable plates include 1.2% R2A agar, 1.2% agar, 1.2% agarose, and 1.2% agarose with 0.3 g/L pyruvate.

In some embodiments, ammonia oxidizing bacteria may be cultured in organic free media. One advantage of using organic free media is that it lacks substrate for heterotrophic bacteria to metabolize except for that produced by the autotrophic bacteria. Another advantage of using the as-grown culture is that substantial nitrite accumulates in the culture media, and this nitrite is also inhibitory of heterotrophic bacteria and so acts as a preservative during storage.

In some embodiments, an ammonia oxidizing bacteria with improved, e.g. optimized, properties is produced by an iterative process of propagation and selecting for desired properties. In some embodiments, the selection and propagation are carried out simultaneously. In some embodiments, the selection is carried out in a reaction medium (e.g., complete N. europaea medium) comprising 50 mM, 75 mM, 100 mM, 125 mM, 150 mM, 175 mM, 200 mM, 225 mM, 250 mM, 275 mM, or 300 mM NH₄⁺, e.g., at least 200 mM NH₄⁺. In some embodiments, the period of propagation and/or selection is at least 1, 2, 3, or 6 months. In embodiments, the period of propagation and/or selection is at least 1, 2, 4, 6, 8, or 10 years.

In some aspects, the ammonia oxidizing bacteria are manufactured on a commercial scale. In some embodiments, commercial scale refers to a liquid culturing method with a culture medium volume of at least 10,000, 20,000, 30,000, 50,000, or 100,000 liters (L). In some embodiments, the bacteria are produced in a bioreactor. The bioreactor may maintain the bacteria at a constant temperature, e.g., about 26-30 degrees Celsius using, for example a thermal jacket for insulation, a temperature sensor, and a heating or cooling element. The bioreactor may have an apparatus for stirring the culture to improve distribution of nutrients like ammonia, urea, oxygen, carbon dioxide, and various minerals. The bioreactor may also have an inlet tube for addition of new medium, and an outlet tube for collection of cells. The bioreactor may also have an aerator for distributing oxygen and/or carbon dioxide to the culture. The bioreactor may be, e.g., a batch reactor, a fed batch reactor, or a continuous reactor. In some embodiments, commercial scale production of ammonia oxidizing bacteria yields a batch of 1,000 to 100,000 L per day at about 10¹² CFU/liter. The commercial scale production may yield e.g., a batch of 1,000-5,000, 5,000-10,000, 10,000-50,000, or 50,000-100,000 L/day. The commercial scale production may yield e.g., a batch of 1,000-5,000, 5,000-10,000, 10,000-50,000, or 50,000-100,000 L per batch. In some embodiments, the yield is at a concentration of at least 10⁸, 10⁹, 10¹⁰, 10¹¹, 2×10¹¹, 5×10¹¹, or 10¹², or about 10¹⁰-10¹¹, 10¹¹-10¹², 10¹²-10¹³, or 10¹³-10¹⁴ CFU/L. In some embodiments, the yield is at a concentration of at least 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/ml.

In some embodiments, typically including commercial scale production, quality control (QC) testing steps are carried out. The general steps of QC may comprise, 1) culturing ammonia oxidizing bacteria, 2) performing a testing step on the culture or an aliquot thereof, and 3) obtaining a value from the testing step, and optionally: 4) comparing the obtained value to a reference value or range of acceptable values, and 5) if the obtained value meets the acceptable reference value or range, then classifying the culture as acceptable, and if the obtained value does not meet the acceptable reference value or range, then classifying the culture as unacceptable. If the culture is classified as acceptable, the culture may, e.g., be allowed to continue growing and/or may be harvested and added to a commercial product. If the culture is classified as unacceptable, the culture may, e.g., be safely disposed of or the defect may be remedied.

The testing step may comprise measuring the optical density (OD) of the culture. OD is measured in a spectrophotometer, and provides information on the amount of light transmitted through the sample as distinguished from light absorbed or scattered. In some embodiments, the OD600 (e.g., optical density of light with a wavelength of 600 nm) may be determined. This measurement typically indicates the concentration of cells in the medium, where a higher optical density corresponds to a higher cell density.

The testing step may comprise measuring the pH of the culture. The pH of an ammonia oxidizing bacteria culture indicates the rate of nitrogen oxidation, and can also indicate whether the culture comprises a contaminating organism. pH may be measured using, e.g., a pH-sensing device comprising a electrode (such as a hydrogen electrode, quinhydron-Electrode, antimony electrode, glass electrode), a pH-sensing device comprising a semiconductor, or a color indicator reagent such as pH paper.

In certain embodiments, producing the ammonia oxidizing bacteria comprises carrying out various quality control steps. For instance, one may test the medium in which the ammonia oxidizing bacteria is grown, e.g., to determine whether it has an appropriate pH, whether it has a sufficiently low level of waste products, and/or whether it has a sufficiently high level or nutrients. One may also test for the presence of contaminating organisms. A contaminating organism is typically an organism other than ammonia oxidizing bacteria, for instance an organism selected from Microbacterium sp., Alcaligenaceae bacterium, Caulobacter sp., Burkodelia multivorans, Escherichia coli, Klebsiella pneumoniae, and Staphylococcus aureus. One may test for contaminants by, e.g., extracting DNA, amplifying it, and sequencing a conserved gene such as 16S rRNA. One may also test for contaminants by plating culture on agar plates and observing colony morphology. Ammonia oxidizing bacteria typically forms red colonies, so non-red colonies are often indicative of contaminating organisms.

### Methods of Testing Product for Ammonia Oxidizing Bacteria and Quality Control Methods

In some embodiments, methods of evaluating a preparation are provided. The method may comprise evaluating a preparation for the presence of, e.g., level, of ammonia oxidizing bacteria. The ammonia oxidizing bacteria may be of the genus selected from the group consisting of Nitrosomonas, Nitrosococcus, Nitrosospria, Nitrosocystis, Nitrosolobus, Nitrosovibrio, and combinations thereof.

The preparation may be a cosmetic product or a therapeutic product, or may be in the process of being prepared or manufactured as a cosmetic product or a therapeutic product. The preparation may be disposed in or provided as a cosmetic product or a therapeutic product. The preparation may be supplied to, e.g., a manufacturer, healthcare company, or consumer product company that may use the preparation or convert the preparation into a therapeutic product or a consumer product.

The methods of evaluating a preparation may be provided to evaluate, e.g., a natural product, a product that has been manufactured, a product that may naturally contain ammonia oxidizing bacteria, or one in which ammonia oxidizing bacteria have been added or will be added.

The method may comprise measuring an amount or a concentration of ammonia oxidizing bacteria in the product to provide a value. This may be accomplished by measuring the pH of the preparation. The pH of an ammonia oxidizing bacteria preparation may indicate the rate of nitrogen oxidation, and may also indicate whether the preparation comprises a contaminating organism. pH may be measured using, e.g., a pH-sensing device comprising a electrode (such as a hydrogen electrode, quinhydron-Electrode, antimony electrode, glass electrode), a pH-sensing device comprising a semiconductor, or a color indicator reagent such as pH paper. In some embodiments, a sample of the preparation may be cultured to provide an ammonia oxidizing culture, that may then be tested by measuring the pH of the culture.

Measuring an amount or a concentration of ammonia oxidizing bacteria in the product to provide a value may be accomplished by measuring the rate of oxidation of ammonia, ammonium ions, and/or urea to provide nitrite, e.g., a rate of convension of ammonia, ammonium ions, and/or urea to intrite, or measuring a level of nitrite in a sample. Measuring the presence or level of nitrite in the preparation may also accomplished to provide a value. In some embodiments, measuring an amount or concentration of protein in the product may also be accomplished to provide a value. The amount or concentration of total protein in a sample may be used to calculate the concentration of ammonia oxidizing bacteria.

In some embodiments, the methods of the present disclosure comprise classifying the preparation as requiring addition of ammonia oxidizing bacteria, or as not requiring addition of ammonia oxidizing bacteria. This may be accomplished by evaluating the value measured, as discussed above, to determine whether it is within or outside a pre-determined range of values.

The value may be compared to a range of values corresponding to the pre-determined range of values, e.g., a range of amounts of ammonia oxidizing bacteria, e.g., a range of concentrations of ammonia oxidizing bacteria. It then may be determined if the value is in the range of values corresponding to the pre-determined range of values. If the value is in the range of values corresponding to the pre-determined range of values, the product may be classified as accepted. This may mean that the product may be suitable for manufacturing, packaging, placing into commerce, supplying or selling to, e.g., a manufacturer, a healthcare company, consumer product company, e.g., a cosmetic company or beauty supply company, a health care provider, or a consumer.

If the value is outside the range of values corresponding to the pre-determined range of values, the product may be classified as not accepted. If the value is outside the range of values corresponding to the pre-determined range of amounts or concentrations of ammonia oxidizing bacteria, the preparation may be further processed or discarded. If the preparation is further processed, the preparation may be diluted, if the amount or concentration of ammonia oxidizing bacteria is higher than the pre-determined range of amounts or concentrations of ammonia oxidizing bacteria. If the preparation is further processed, additional components may be added, for example, if the value is outside the range of values corresponding to the pre-determined range of values, e.g., below the range of values corresponding to the pre-determined range.

In some embodiments in which the value is outside the range of values corresponding to the pre-determined range of values, an amount or concentration of ammonia oxidizing bacteria may be added to the preparation. In other embodiments, addition of one or more of ammonia, ammonium ions, and urea may be performed. In some embodiments, a combination of one or more of ammonia oxidizing bacteria, ammonia, ammonium ions, and urea may be added.

Further evaluation may be performed to determine if the preparation may be classified as accepted, or not accepted, as described above.

The product may be further evaluated to determine if contaminating organisms, e.g., pathogenic organisms, are present. This may be accomplished by testing the pH of the preparation. Pathogenic organisms may include Staphylococcus aureus (S. aureus), Psuedonomas aeruginosa (P. aeruginosa), Streptococcus pyogenes (S. pyogenes), Acinetobacter baumannii (A. baumannii), Propionibacteria, and Stenotrophomonas, and combinations thereof. This may also be accomplished by ingsin bioburden tests, e.g., plating a sample of the preparation on LB agar plates and culturing them at 30-37° C. for a period of time, e.g., about 24 hours or more, to determine the presence of pathogenic organisms. This may also be accomplished by using 15S rRNA sequencing to determine pathogens that may be present.

In some embodiments, a sample from the product may be selected and testing may be conducted on the sample.

The range of values corresponding to a pre-determined range of concentrations of ammonia oxidizing bacteria may be between about 10⁸ CFU/L to about 10¹⁴ CFU/L. In certain aspects, the range of values corresponding to the pre-determined range of amounts or concentrations of ammonia oxidizing bacteria is less than about 10⁸, or between about 10⁸-10⁹, 10⁹-10¹⁰, 10¹⁰-10¹¹, 10¹¹-10¹², 10¹²-10¹³, or 10¹³-10¹⁴ CFU/L.

The range of values corresponding to a pre-determined range of concentrations of ammonia oxidizing bacteria may be between about 10⁸ CFU/ml to about 10¹⁴ CFU/ml. In certain aspects, the range of values corresponding to the pre-determined range of amounts or concentrations of ammonia oxidizing bacteria is less than about 10⁸, or between about 10⁸-10⁹, 10⁹-10¹⁰, 10¹⁰-10¹¹, 10¹¹-10¹², 10¹²-10¹³, or 10¹³-10¹⁴ CFU/ml.

In certain aspects, the preparation may comprise between about 1×10⁹ CFU to about 10×10⁹ CFU.

The range of values corresponding to a pre-determined range of amounts of ammonia oxidizing bacteria may comprise between about 0.1 milligrams (mg) and about 1000 mg of ammonia oxidizing bacteria. In certain aspects, the preparation may comprise between about 50 mg and about 1000 mg of ammonia oxidizing bacteria. The range may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

Also provided herein are methods of identifying an active strain of AOB, where an active strain of AOB generates a detectable level of nitro-fatty acids or nitro-hydrocarbons. In embodiments, the method comprises incubating a candidate strain of AOB with an unsaturated fatty acid or unsaturated hydrocarbon, e.g., in vitro, e.g., in a container such as a plate, dish, flask, or test tube. In embodiments, the incubation is performed for 1 hour to 7 days (e.g., 1-2 h, 2-4 h, 4-6 h, 6-12 h, 12-24 h, 1-2 days, 2-3-days, 3-4 days, 4-5 days, 5-6 days, or 6-7 days). In embodiments, the incubation is performed at a temperature of 4° C. to 35° C. (e.g., 4° C. to 10° C., 10° C. to 20° C., 20° C. to 30° C., or 30° C. to 35° C. After a time period of 1 hour to 7 days (e.g., 1-2 h, 2-4 h, 4-6 h, 6-12 h, 12-24 h, 1-2 days, 2-3-days, 3-4 days, 4-5 days, 5-6 days, or 6-7 days), the level of nitro-fatty acid or nitro-hydrocarbon is determined, e.g., using standard methods in the art. See, e.g., Woodcock et al. Nitrated fatty acids: Synthesis and Measurement. Free Radic. Biol. Med. 59 (2013):14-26; and Bonacci et al. J. Am. Soc. Mass Spectrom. 22.9 (2011):1534-51, both incorporated herein by reference. For example, the level of nitro-fatty acid or nitro-hydrocarbon is determined using methods such as gas chromatography (GC), liquid chromatography (LC), reverse phase high performance liquid chromatography (RP-HPLC), mass spectrometry (MS), GC-MS, or LC-MS. Generation of a level of nitro-fatty acid or nitro-hydrocarbon above a control level indicates that the candidate AOB strain is active. In embodiments, a control level is a level of the nitro-fatty acid or nitro-hydrocarbon detected by performing an assay described above using dead AOB, or using non-ammonia oxidizing bacteria. In embodiments, a control level is an undetectable level of nitro-fatty acid or nitro-hydrocarbon.

The method of evaluating of the present disclosure may comprise evaluating the preparation from a first batch, e.g., a first batch for commercial release. The preparation from a second batch may be evaluated, e.g., a second batch for commercial release. One or more additional batches, e.g., a third, fourth, fifth, sixth, seventh, eighth, ninth, tenth, fifteen twentieth, hundredth, or more, batches may be evaluated, e.g., for commercial release. Multiple batches may be evaluated, and additional batches may be evaluated at pre-determined time or batch intervals in order to, e.g., evaluate the consistency of preparations over time, and over manufacturing batches, which also may be referred to as lots.

The method of the present disclosure may comprise evaluating the preparation from a plurality of batches to determine if each batch within the plurality meets the pre-determined range of amounts or concentrations of ammonia oxidizing bacteria.

The batches may comprise releasing the plurality of batches. The plurality of batches, e.g., at least one batch, may be commercially released to, e.g., a manufacturer, a healthcare company, consumer product company, e.g., a cosmetic company or beauty supply company, a health care provider, or a consumer.

The batches may be prepared simultaneously or consecutively. Simultaneously may mean that the preparation of one batch is still occurring when the preparation of the second batch begins, so that there is overlap. This may be referred to as “simultaneous,” “concomitant,” or “concurrent” preparation. Consecutively may mean that the preparation of one batch ends before the preparation of the second batch. This may be referred to herein as “successive,” “sequential,” or “consecutive” preparation.

Compositions may be provided, e.g., preparations or formulations, from the methods of evaluating as described above. The compositions may comprise one or more excipients as described throughout this disclosure, and may be suitable for one of topical, nasal, pulmonary, and gastrointestinal administration. The compositions may be substantially free of other organisms. They may be disposed in, or provided as a powder, cosmetic, cream, stick, aerosol, salve, wipe, or bandage, and them may further comprise a moisturizing agent, deodorizing agent, scent, colorant, insect repellant, cleansing agent, or UV-blocking agent, as described herein. In some embodiments, the compositions may comprise an organism selected from the group consisting of Lactobacillus, Streptococcus, Bifidobacter, and combinations thereof.

The compositions described herein, e.g., provided by the methods of evaluating, may be useful as a treatment or prevention of a skin disorder, a treatment or prevention of a disease or condition associated with low nitrite levels, a treatment or prevention of body odor, a treatment to supply nitric oxide to a subject, or a treatment to inhibit microbial growth.

They may be useful for treatment of at least one of HIV, dermatitis, infection in a diabetic foot ulcer, atopic dermatitis, acne, eczema, contact dermatitis, allergic reaction, psoriasis, uticaria, rosacea, skin infections, vascular disease, vaginal yeast infection, a sexually transmitted disease, heart disease, atherosclerosis, baldness, leg ulcers secondary to diabetes or confinement to bed, angina, particularly chronic, stable angina pectoris, ischemic diseases, congestive heart failure, myocardial infarction, ischemia reperfusion injury, laminitis, hypertension, hypertrophic organ degeneration, Raynaud's phenomenon, fibrosis, fibrotic organ degeneration, allergies, autoimmune sensitization, end stage renal disease, obesity, impotence, pneumonia, primary immunodeficiency, epidermal lysis bulosa, or cancer.

In certain aspects the compositions described herein may be useful for treatment of at least one of an infection in a diabetic foot ulcer, atopic dermatitis, acne, eczema, psoriasis, uticaria, rosacea, and skin infections.

The compositions described herein, e.g., provided by the methods of evaluating, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, the compositions described herein, e.g., provided by the methods of evaluating, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

### Methods of Evaluating a Subject

Methods of evaluating a subject are provided. The methods may comprise evaluating a subject for application of ammonia oxidizing bacteria. This may comprise a self-evaluation in which a subject evaluates their own condition, e.g., condition of the skin, or their health condition, and evaluate whether to administer or apply ammonia oxidizing bacteria to their self. This may also comprise evaluation of a subject by another, e.g., a healthcare professional, to evaluate the subject's condition, e.g., condition of the skin, or their health condition, and evaluate whether to administer or apply ammonia oxidizing bacteria to the subject, or advise the subject or another individual to administer or apply ammonia oxidizing bacteria to the subject. The methods may be used to determine if the subject qualifies for application of ammonia oxidizing bacteria.

The methods may comprise assessing the subject for a skin condition. Responsive to the assessing, one of the following may be performed: (1) classifying the subject as requiring ammonia oxidizing bacteria; and (2) administering ammonia oxidizing bacteria. This may provide a method for determining if a subject qualifies for application of ammonia oxidizing bacteria. The subject may perform the step of assessing, or another individual may perform the step of assessing. In some embodiments, the subject may be classified as not requiring ammonia oxidizing bacteria. In these embodiments, the subject may not be administered ammonia oxidizing bacteria.

The skin disorder or skin condition that may be assessed may be selected from the group consisting of dermatitis, general itchiness, infection in a diabetic foot ulcer, atopic dermatitis, acne, eczema, contact dermatitis, allergic reaction, psoriasis, uticaria, rosacea, skin infections, leg ulcers secondary to diabetes or confinement to bed, epidermal lysis bulosa, and allergies.

The method may comprise obtaining a sample from a surface of the subject. This may involve any method of obtaining a sample from a surface of the subject, e.g., contacting the surface of the subject, e.g., the skin, with a wipe, cloth, cotton ball, cotton swab, gauze, towel, towelette, or other material in order to obtain a sample of bacteria from the surface. Once the sample has been taken, the surface of the material may be tested in order to determine if a component is present.

Depending on the component to be tested, a resulting recommended treatment may be suggested. For example, if the presence of ammonia oxidizing bacteria is determined, then no action may be taken. Alternatively, additional ammonia oxidizing bacteria, ammonia, ammonium ions, or urea may be administered or applied at a pre-determined concentration or amount to the subject, e.g., a surface of the subject, e.g., the skin. This may depend on whether the presence or level detected is determined as adequate, e.g., in a range of values that would be appropriate for treatment of a condition of a subject.

If presence of ammonia, ammonium ions, or urea is determined, then an action may or may not be taken. For example, if presence of ammonia, ammonium ions, or urea is determined, at least one of ammonia oxidizing bacteria, ammonia, ammonium ions, and urea, may or may not be administered or applied at a pre-determined concentration or amount to the subject, e.g., a surface of the subject, e.g., the skin.

If the presence of one or more pathogenic bacteria is determined, then an action may or may not be taken. For example, if presence of pathogenic bacteria is determined, at least one of ammonia oxidizing bacteria, ammonia, ammonium ions, and urea, may or may not be administered or applied at a pre-determined concentration or amount to the subject, e.g., a surface of the subject, e.g., the skin.

If the presence of one or more of nitrite and nitric oxide is determined, then an action may or may not be taken. For example, if presence of nitrite and/or nitric oxide is determined, at least one of ammonia oxidizing bacteria, ammonia, ammonium ions, and urea, may or may not be administered or applied at a pre-determined concentration or amount to the subject, e.g., a surface of the subject, e.g., the skin.

A level of a component that is present may be determined. This may be a quantitative or qualitative determination. For example, an amount or concentration of a component may be determined. This amount or concentration may be used to determine whether administration or application of ammonia oxidizing bacteria is required or desired, or whether administration or application of other compounds, e.g., ammonia, ammonium ions or urea is required or desired.

The determined amount or concentration may be compared to a range of values corresponding to a pre-determined range of amount or concentration. The amount or concentration may be determined to be within or outside of the range of values corresponding to the pre-determined range.

In certain aspects, if the amount or concentration is in the range of values corresponding to the pre-determined range, the product may be classified as accepted. In certain other aspects, if the amount or concentration is outside the range of values corresponding to the pre-determined range, the product may be classified as accepted.

In certain aspects, if the amount or concentration is in the range of values corresponding to the pre-determined range, the product may be classified as not accepted. In certain other aspects, if the amount or concentration is outside the range of values corresponding to the pre-determined range, the product may be classified as not accepted.

If the amount or concentration is classified as not accepted, administration or application of at least one of ammonia oxidizing bacteria, ammonia, ammonium ions, and urea may be performed. If the amount or concentration is classified as accepted, no administration or application of at least one of ammonia oxidizing bacteria, ammonia, ammonium ions, and urea may be performed.

The pre-determined range of concentrations of ammonia oxidizing bacteria may be between about 10⁸ CFU/L and about 10¹⁴ CFU/L. In certain aspects, the pre-determined range of concentrations of ammonia oxidizing bacteria is less than about 10⁸ or between about 10⁸-10⁹, 10⁹-10¹⁰, 10¹⁰-10¹¹, 10¹¹-10¹², 10¹²-10¹³, or 10¹³-10¹⁴ CFU/L.

The pre-determined range of concentrations of ammonia oxidizing bacteria may be between about 10⁸ CFU/ml and about 10¹⁴ CFU/ml. In certain aspects, the pre-determined range of concentrations of ammonia oxidizing bacteria is less than about 10⁸ or between about 10⁸-10⁹, 10⁹-10¹⁰, 10¹⁰-10¹¹, 10¹¹-10¹², 10¹²-10¹³, or 10¹³-10¹⁴CFU/ml.

The pre-determined range of amounts of ammonia oxidizing bacteria may comprise between about 0.1 milligrams (mg) and about 1000 mg of ammonia oxidizing bacteria. In certain aspects, the pre-determined range may be between about 50 mg and about 1000 mg of ammonia oxidizing bacteria. The range may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

The concentration of ammonia oxidizing bacteria administered or applied may be between about 10⁸ CFU/L to about 10¹⁴ CFU/L. In certain aspects, the concentration of ammonia oxidizing bacteria administered or applied may be less than about 10⁸ or between about 10⁸-10⁹, 10⁹-10¹⁰, 10¹⁰-10¹¹, 10¹¹-10¹², 10¹²-10¹³, or 10¹³-10¹⁴ CFU/L.

The concentration of ammonia oxidizing bacteria administered or applied may be between about 10⁸ CFU/ml to about 10¹⁴ CFU/ml. In certain aspects, the concentration of ammonia oxidizing bacteria administered or applied may be less than about 10⁸ or between about 10⁸-10⁹, 10⁹-10¹⁰, 10¹⁰-10¹¹, 10¹¹-10¹², 10¹²-10¹³, or 10¹³-10¹⁴ CFU/ml.

The amount of ammonia oxidizing bacteria administered or applied may comprise between about 0.1 milligrams (mg) and about 1000 mg of ammonia oxidizing bacteria. In certain aspects, the amount administered or applied may comprise between about 50 mg and about 1000 mg of ammonia oxidizing bacteria. The amount administered or applied may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

### Methods of Preparing AOB Product

Methods of preparing a product comprising ammonia oxidizing bacteria is provided. The preparation may or may not involve adding additional, e.g., exogenous ammonia oxidizing bacteria to provide an ammonia oxidizing bacteria-containing product, or supplementing a product with additional ammonia oxidizing bacteria.

This method may comprise measuring an amount or a concentration of ammonia oxidizing bacteria in a preparation. The measurement may provide a value. Responsive to that value, the preparation may be classified as requiring ammonia oxidizing bacteria or not requiring ammonia oxidizing bacteria.

The method may further comprise, if the preparation is classified as requiring ammonia oxidizing bacteria, adding an amount or concentration of ammonia oxidizing bacteria. In addition, or in the alternative, the method may further comprise adding an amount or concentration of at least one of ammonia, ammonium ions, and urea to the preparation. This may be based on the value that was measured. The addition of one or more of these components may provide a product that may be released into commerce.

In certain aspects, responsive to the value measure, the preparation may be passed into a next step of releasing into commerce the product, without addition of ammonia oxidizing bacteria.

The product (including preparations) provided by the methods discussed herein may be packaged into a package. e.g., a container, which may then be placed in commerce. The packaging may comprise an aseptic or sterile compartment. The packaging may be substantially free of other organisms. The packaging may be substantially preservative-free or contain preservative or other components, e.g., excipients or chelator as discussed in this disclosure.

The products may be provided in a container, delivery system, or delivery device. The ammonia oxidizing bacteria may be in a growth state or a storage state. Other components, such as ammonia, ammonium ions, and/or urea may be included in the container. The additional components may be in a separate compartment or chamber as the ammonia oxidizing bacteria, and upon actuation of the container, delivery system, or delivery device, or upon delivery of the contents, the ammonia oxidizing bacteria may contact, e.g., be mixed, with the other components of the container.

### Fortifying Natural Products

Natural products may be fortified by the present disclosure. Natural product or fortified natural product may comprise at least one of mud, water, food-derived products, plant-derived products, extracts, and oils. The natural products may be used in cosmetic and therapeutic applications, e.g., in cosmetic products, e.g., therapeutic products, e.g., spa treatments.

The natural product or the fortified natural product may be incorporated into at least one of a powder, cream, lotion, wrap, scrub, eye mask, facial mask, body mask, aerosol, spray, salve, wipe, stick, bandage, or soak.

The natural product or fortified natural product may be provided as, or is disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

The natural products may be fortified with ammonia oxidizing bacteria. In addition, or in the alternative, the natural product may be fortified with ammonia, ammonium ions, and/or urea. In some embodiments, the natural product is not fortified with ammonia, ammonium ions, or urea. In embodiments, the natural product is fortified with a nitro-fatty acid or nitro-hydrocarbon. In embodiments, the natural product is fortified with a nitro-fatty acid or nitro-hydrocarbon in addition to ammonia oxidizing bacteria. In certain aspects, the natural products may naturally comprise ammonia oxidizing bacteria (that is, obtained from natural sources without the addition of ammonia oxidizing bacteria, e.g., without human intervention). In certain aspects, the natural products may naturally comprise ammonia, ammonium ions, and/or urea. The ammonia oxidizing bacterial may be in a growth state or a storage state. In embodiments, the AOB is dead, e.g., at least 10%, 20%, 30%, 40%, 50%, 60%, 70%, 80%, 90%, 95%, or 99% (by weight) of the AOB in the fortified natural product is dead.

Fortifying natural products may comprise measuring an amount or a concentration of ammonia oxidizing bacteria in the natural product. The measurement may provide a value. Responsive to that value, the natural product may be classified as requiring ammonia oxidizing bacteria, or classifying the natural product as not requiring ammonia oxidizing bacteria.

An amount or a concentration of ammonia oxidizing bacteria may be added to the natural product. This may be performed responsive to the value measured, and/or in response to the classification as described above. This may provide a fortified natural product.

An amount or a concentration of ammonia, ammonium ions, and urea may be added to the natural product. This may be performed responsive to the value measured, and/or in response to the classification as described above. This may provide a fortified natural product.

In response to the value measured, the natural product may be passed to the next step of releasing the natural product into commerce without addition of ammonia oxidizing bacteria.

A fortified natural product may also be passed to the next step of releasing the fortified natural product into commerce.

The product, e.g., the natural product or the fortified natural product (including preparations comprising one or more natural products) provided by the methods discussed herein may be packaged into a package. e.g., a container, which may then be placed in commerce. The packaging may comprise an aseptic or sterile compartment. The package may be substantially free of other organisms. The packaging may be substantially preservative-free or contain preservative or other components, e.g., excipients or chelator as discussed in this disclosure.

The products may be provided in a container, delivery system, or delivery device. The ammonia oxidizing bacteria may be in a growth state or a storage state. In embodiments, the AOB is dead, e.g., at least 10%, 20%, 30%, 40%, 50%, 60%, 70%, 80%, 90%, 95%, or 99% (by weight) of the AOB in the fortified natural product is dead. Other components, such as ammonia, ammonium ions, and/or urea may be included in the container. The additional components may be in a separate compartment or chamber as the ammonia oxidizing bacteria, and upon actuation of the container, delivery system, or delivery device, or upon delivery of the contents, the ammonia oxidizing bacteria may contact, e.g., be mixed, with the other components of the container.

The weight of the package, container, delivery system, or delivery device, with or without contents may be is less than about 50, 100, 200, 300, 400, 500, 600, 700, 800, 900, 1000, 1500, or 2000 grams.

The package, natural product or the fortified natural product may be provided as, disposed in, or comprise a powder, cosmetic, cream, stick, aerosol, salve, wipe, or bandage.

The range of concentrations of ammonia oxidizing bacteria that the natural product may be fortified with or may comprise may be between about 10⁸ CFU/L to about 10¹⁴ CFU/L. In certain aspects, the range of concentrations of ammonia oxidizing bacteria is less than about 10⁸ or between about 10⁸-10⁹, 10⁹-10¹⁰, 10¹⁰-10¹¹, 10¹¹-10¹², 10¹²-10¹³, or 10¹³-10¹⁴ CFU/L.

The range of concentrations of ammonia oxidizing bacteria that the natural product may be fortified with or may comprise may be between about 10⁸ CFU/ml to about 10¹⁴ CFU/ml. In certain aspects, the range of concentrations of ammonia oxidizing bacteria is less than about 10⁸ or between about 10⁸-10⁹, 10⁹-10¹⁰, 10¹⁰-10¹¹, 10¹¹-10¹², 10¹²-10¹³, or 10¹³-10¹⁴CFU/ml.

The range of amounts of ammonia oxidizing bacteria that the natural product may be fortified with or may comprise may be between about 0.1 milligrams (mg) and about 1000 mg of ammonia oxidizing bacteria. In certain aspects, the range may be between about 50 mg and about 1000 mg of ammonia oxidizing bacteria. The range may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

In embodiments, the natural product is fortified with 0.0005 to 2% by weight, e.g., 0.001 to 1% by weight (e.g., 0.001 to 0.01%, 0.005 to 0.05%, 0.01 to 0.1%, 0.05 to 0.5%, 0.1 to 1%, or 0.5% to 1% by weight) of a nitro-fatty acid or nitro-hydrocarbon, e.g., nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

In some embodiments, the natural product or fortified natural product may comprise a mass ratio of ammonia oxidizing bacteria to an excipient, e.g., a pharmaceutically acceptable excipient or a cosmetically acceptable excipient in a range of about 0.1 grams per liter to about 1 gram per liter. The natural product or fortified natural product may comprise a mass ratio of ammonia oxidizing bacteria to an excipient in a range of about 0.1-0.2, 0.2-0.3, 0.1-0.5, 0.2-0.7, 0.5-1.0, or 0.7-1.0 grams per liter.

Compositions Comprising Ammonia Oxidizing Bacteria and/or a Nitro-Hydrocarbon or Nitro-Fatty Acid

The present disclosure provides, inter alia, compositions comprising ammonia oxidizing bacteria, e.g., a preparation of ammonia oxidizing bacteria, or a purified preparation of ammonia oxidizing bacteria, e.g., a natural product, or a fortified natural product, or a cosmetic or pharmaceutical composition.

The present disclosure also provides compositions comprising a nitro-fatty acid or nitro-hydrocarbon, e.g., manufactured by using AOB, e.g., manufactured using methods described herein.

In embodiments, the composition comprises an AOB in combination with a nitro-hydrocarbon or a nitro-fatty acid. In embodiments, the composition comprises an AOB and does not comprise a nitro-hydrocarbon or nitro-fatty acid.

The compositions can be provided in a cosmetic product or a therapeutic pharmaceutical product.

In some embodiments, the preparation comprises at least one of ammonia, ammonium ions, and urea. In other embodiments, the preparation does not comprise ammonia, ammonium ions, or urea. In embodiments, the composition comprises a nitro-hydrocarbon or nitro-fatty acid described herein. For example, the composition comprises nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

In embodiments, the composition comprises 90% or less (by weight) of AOB, e.g., 90%, 80%, 70%, 60%, 50%, 40%, 30%, 20%, 10%, 5%, 2.5%, 1%, or less (by weight) of AOB. In embodiments, the composition comprises 90% or more (by weight) of AOB, e.g., 90%, 80%, 70%, 60%, 50%, 40%, 30%, 20%, 10%, 5%, 2.5%, 1%, or more (by weight) of AOB.

In embodiments, the composition comprises 90% or less (by weight) of a nitro-hydrocarbon or nitro-fatty acid, e.g., 90%, 80%, 70%, 60%, 50%, 40%, 30%, 20%, 10%, 5%, 2.5%, 1%, 0.5%, 0.1%, 0.05%, 0.01%, 0.005%, 0.001%, or less (by weight) of the nitro-hydrocarbon or nitro-fatty acid. In embodiments, the composition comprises 0.0005 to 5% by weight (e.g., 0.0005 to 0.005%, 0.001 to 0.01%, 0.005 to 0.05%, 0.01 to 0.1%, 0.05 to 0.5%, 0.1 to 1%, or 0.5% to 5% by weight) of a nitro-hydrocarbon or a nitro-fatty acid. In an embodiment, the nitro-fatty acid or the nitro-hydrocarbon is present at a concentration of 0.001 to 1% by weight.

In embodiments, the composition comprises 0.001% or more (by weight) of a nitro-hydrocarbon or nitro-fatty acid, e.g., 0.001%, 0.005%, 0.01%, 0.05%, 0.1%, 0.5%, 1%, 5%, 10%, 15%, 20%, 30%, 40%, 50%, 60%, or more (by weight) of the nitro-hydrocarbon or nitro-fatty acid.

In some embodiments, the composition comprises a therapeutically effective amount of a nitro-fatty acid or nitro-hydrocarbon. In embodiments, the therapeutically effective amount is higher than that found on a skin of a mammal, e.g., human, e.g., that has not been administered an AOB or other exogenous source of nitro-fatty acid or nitro-hydrocarbon. In some embodiments, the composition comprises a concentration of nitro-fatty acid or nitro-hydrocarbon that is higher than that found on a skin of a mammal, e.g., human, that has been topically administered an AOB (e.g., topically applied a composition comprising active AOB), e.g., a topically administered AOB-containing composition that does not also contain exogenous nitro-fatty acids and/or nitro-hydrocarbons.

In some embodiments, the composition comprises ammonia oxidizing bacteria, e.g., one genus of ammonia oxidizing bacteria, or one species of ammonia oxidizing e.g., N. eutropha, and one other type of organism, and no other types of organism. In other examples, the composition has ammonia oxidizing bacteria, or more specifically has one genus of ammonia oxidizing bacteria, or more specifically, having one species of ammonia oxidizing e.g., N. eutropha and 2, 3, 4, 5, 6, 7, 8, 9, or 10 other types of organism, and no other types of organism. Suitable ammonia-oxidizing bacteria include those in the genera Nitrosomonas, Nitrosococcus, Nitrosospira, Nitrosocystis, Nitrosolobus, or Nitrosovibrio.

In some embodiments, one or more other organisms besides ammonia oxidizing bacteria may be included in the composition. For example, an organism of the genus selected from the group consisting of Lactobacillus, Streptococcus, Bifidobacter, and combinations thereof, may be provided in the composition. In some embodiments, the composition is substantially free of other organisms.

In some embodiments, the composition provides conditions that support ammonia oxidizing bacteria viability. For instance, the composition may promote ammonia oxidizing bacteria growth and metabolism or may promote a dormant state (e.g., freezing) or storage state as described herein from which viable ammonia oxidizing bacteria can be recovered. When the composition promotes growth or metabolism, it may contain water and/or nutrients that ammonia oxidizing bacteria consumes, e.g., as ammonium ions, ammonia, urea, oxygen, carbon dioxide, or trace minerals.

In embodiments, the composition comprises live AOB, dead AOB, or a mixture of live and dead AOB. In embodiments, more than 50% (by weight) (e.g., more than 50%, 60%, 70%, 80%, 90%, 95%, or 99%) of the AOB in the composition is dead. In other embodiments, more than 50% (by weight) (e.g., more than 50%, 60%, 70%, 80%, 90%, 95%, or 99%) of the AOB in the composition is alive.

The composition can comprise ammonia oxidizing bacteria at a concentration of between about between about 10⁸ to about 10¹⁴ CFU/L, e.g., at least about 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/L.

In some embodiments, the composition comprises between about 1×10⁹ to about 10×10⁹ CFU/L of AOB, e.g., about 3×10¹⁰ CFU, e.g., 3×10¹⁰ CFU per day; or about 1×10⁹ to about 10×10⁹ CFU, e.g., about 1×10⁹ to about 10×10⁹ CFU per day.

In some embodiments, the composition comprises between about 0.1 milligrams (mg) and about 1000 mg of ammonia oxidizing bacteria. For example, the composition comprises between about 50 mg and about 1000 mg of ammonia oxidizing bacteria. In embodiments, the composition comprises between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg of AOB.

In embodiments, the composition comprises 0.0005 to 5% by weight, e.g., 0.001 to 1% by weight (e.g., 0.001 to 0.01%, 0.005 to 0.05%, 0.01 to 0.1%, 0.05 to 0.5%, 0.1 to 1%, or 0.5% to 1% by weight) of a nitro-fatty acid or nitro-hydrocarbon, e.g., nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, or a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

In some embodiments, the composition may comprise a mass ratio of ammonia oxidizing bacteria to an excipient, e.g., a pharmaceutically acceptable excipient or a cosmetically acceptable excipient in a range of about 0.1 grams per liter to about 1 gram per liter. The preparation may comprise a mass ratio of ammonia oxidizing bacteria to an excipient in a range of about 0.1-0.2, 0.2-0.3, 0.1-0.5, 0.2-0.7, 0.5-1.0, or 0.7-1.0 grams per liter.

The composition can comprise one or more excipients, e.g., one or more pharmaceutically acceptable excipients or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

In embodiments, the composition is present in a pharmaceutical formulation, preparation, or composition, or a cosmetic formulation, preparation, or composition. Accordingly, this disclosure provides a pharmaceutical formulation (preparation or composition) or a cosmetic formulation (preparation or composition) comprising ammonia oxidizing bacteria and/or a nitro-hydrocarbon or nitro-fatty acid and a pharmaceutically acceptable excipient or a cosmetically acceptable excipient. Pharmaceutical compositions and cosmetic compositions may take the form of a formulation as described below.

The pharmaceutical and cosmetic formulations (e.g., preparations or compositions) described herein may include those suitable for oral (e.g., by way of, or for the purposes of depositing in the gastrointestinal tract), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally (nasal) or via the lungs (pulmonary)), rectal and topical (including dermal, transdermal, transmucosal, buccal, sublingual, and intraocular) administration, although the most suitable route may depend upon, for example, the condition and disorder of the recipient.

The formulations (e.g., preparations or compositions) may conveniently be presented in unit dosage form and may be prepared by any of the methods known in the art of pharmacy or cosmetology. Typically, methods include the step of bringing the active ingredient (e.g., ammonia oxidizing bacteria) into association with a pharmaceutical or a cosmetic carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of ammonia oxidizing bacteria; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988.

The formulations, compositions, or preparations, can, for example, be administered in a form suitable for immediate release or controlled (extended) release. Suitable examples of sustained-release systems include suitable polymeric materials, for example semi-permeable polymer matrices in the form of shaped articles, e.g., films, or microcapsules; suitable hydrophobic materials, for example as an emulsion in an acceptable oil; or ion exchange resins. Controlled (sustained)-release systems may be administered orally; rectally; parenterally; intracistemally; intravaginally; intraperitoneally; topically, for example as a powder, ointment, gel, drop or transdermal patch; bucally; or as a spray.

Preparations for administration can be suitably formulated to give controlled release of nitro-hydrocarbon, nitro-fatty acid, and/or ammonia oxidizing bacteria. For example, the formulations, preparations, or compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486. The preparation may comprise a controlled release material.

Exemplary compositions, e.g., as a preparation, may include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, surfactants, glidants, flavors, coloring agents and stabilizers may also be added for ease of fabrication and use. The surfactant may be a zwitterionic surfactant, a non-ionic surfactant, or an anionic surfactant.

Excipients, such as surfactants that may be used with embodiments of the present disclosure may include one or more of cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's Castile baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K). and combinations thereof. Dr. Bronner's Castile soap comprises water, organic coconut oil, potassium hydroxide, organic olive oil, organic fair deal hemp oil, organic jojoba oil, citric acid, and tocopherol.

In some embodiments, surfactants may be used with ammonia oxidizing bacteria in amounts that allow nitrite production to occur. In some embodiments, the preparation may have less than about 0.0001% to about 10% of surfactant. In some embodiments, the preparation may have between about 0.1% and about 10% surfactant. In some embodiments, the concentration of surfactant used may be between about 0.0001% and about 10%. In some embodiments, the preparation may be substantially free of surfactant.

In some embodiments, the formulation, e.g., preparation, may include other components that may enhance effectiveness of a nitro-hydrocarbon or nitro-fatty acid, or enhance the effectiveness of ammonia oxidizing bacteria, or enhance a treatment of an indication.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., ammonia oxidizing bacteria, e.g. a preparation of ammonia oxidizing bacteria, e.g., an excipient. In some embodiments, the preparation may be substantially free of chelator.

Formulations (e.g., preparations, including natural products and fortified natural products) may also contain anti-oxidants, buffers, bacteriostats that prevent the growth of undesired bacteria, solutes, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition, e.g., a preparation, may also contain minor amounts of non-toxic auxiliary substances, such as wetting or emulsifying agents, surfactants, preservatives, and pH buffering agents and the like, for example sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

In some embodiments, the preparation may be substantially free of one or more of the compounds or substances listed in the disclosure.

Exemplary compositions for aerosol administration include solutions in saline, which can contain, for example, benzyl alcohol or other suitable preservatives, absorption promoters to enhance bioavailability, and/or other solubilizing or dispersing agents. Conveniently in compositions for aerosol administration the ammonia oxidizing bacteria and/or nitro-hydrocarbon/nitro-fatty acid may be delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin can be formulated to contain a powder mix of the ammonia oxidizing bacteria and/or nitro-hydrocarbon/nitro-fatty acid and a suitable powder base, for example lactose or starch. In certain embodiments, ammonia oxidizing bacteria and/or nitro-hydrocarbon/nitro-fatty acid is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations may be presented with carriers such as cocoa butter, synthetic glyceride esters or polyethylene glycol. Such carriers are typically solid at ordinary temperatures, but liquefy and/or dissolve at body temperature to release the ammonia oxidizing bacteria and/or nitro-hydrocarbon/nitro-fatty acid.

Exemplary compositions for topical administration include a topical carrier such as Plastibase (mineral oil gelled with polyethylene). In some aspects, the composition, e.g., preparation, and/or excipient may be in the form of one or more of a liquid, a solid, or a gel. For example, liquid suspensions may include, but are not limited to, water, saline, phosphate-buffered saline, or an ammonia oxidizing storage buffer.

Gel formulations may include, but are not limited to agar, silica, polyacrylic acid (for example Carbopol®), carboxymethul cellulose, starch, guar gum, alginate or chitosan.

In some embodiments, the formulation, e.g., preparation, may be supplemented with an ammonia source including, but not limited to one or more of ammonia, ammonium ions, e.g., ammonium chloride or ammonium sulfate, and urea.

In some embodiments, the composition, e.g., preparation, is formulated to improve NO penetration into the skin. A gel-forming material such as KY jelly or various hair gels would present a diffusion barrier to NO loss to ambient air, and so improve the skin's absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations, e.g., preparations, as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of ammonia oxidizing bacteria, nitro-hydrocarbon, and/or nitro-fatty acid.

In embodiments, a suitable dosage formulation (e.g., topical formulation) comprises about 3×10⁶ to 3×10⁷ AOB cells/cm² of skin.

A therapeutically effective amount of ammonia oxidizing bacteria and/or nitro-hydrocarbon/nitro-fatty acid may be administered as a single pulse dose, as a bolus dose, or as pulse doses administered over time. Thus, in pulse doses, a bolus administration of ammonia oxidizing bacteria and/or nitro-hydrocarbon/nitro-fatty acid is provided, followed by a time period wherein ammonia oxidizing bacteria and/or nitro-hydrocarbon/nitro-fatty acid is administered to the subject, followed by a second bolus administration. In specific, non-limiting examples, pulse doses are administered during the course of a day, during the course of a week, or during the course of a month.

In some embodiments, the composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the composition is administered for an indefinite period of time, e.g, greater than one year, greater than 5 years, greater than 10 years, greater than 15 years, greater than 30 years, greater than 50 years, greater than 75 years.

In some embodiments, the composition, may be applied a pre-determined number of times per day. This may be based, for example, at least in part, on the severity of the condition or disease, the response to the treatment, the dosage applied and the frequency of the dose. For example, the preparation may be applied about 1, 2, 3, 4, 5, 6, 7, 8, 9, 10, 11, 12, 13, 14, 15, 16, 17, 18, 19, 20, 21, 22, 23, 24 times per day.

In some embodiments, the composition may be applied one time per day. In other embodiments, the composition may be applied two times per day. In some embodiments, the composition may be applied a first pre-determined amount for a certain number of days, and a second pre-determined amount for a certain subsequent number of days. In some embodiments, the composition may be applied for about 16 days.

Ammonia oxidizing bacteria may be associated with a variety of consumer products, and examples of such products are set out below. In some embodiments, the ammonia oxidizing bacteria associated with a product is admixed with the product, for example, spread evenly throughout the product, and in some embodiments, the ammonia oxidizing bacteria associated with a product is layered on the product.

In some embodiments, the composition is associated with a powder. Powders are typically small particulate solids that are not attached to each other and that can flow freely when tilted. Exemplary powders for consumer use include talcum powder and some cosmetics (e.g., powder foundation, including pressed powders). Other powders may be contemplated for use in conjunction with ammonia oxidizing bacteria and/or nitro-hydrocarbon/nitro-fatty acid systems and methods of the present disclosure.

In some embodiments, the composition is associated with a cosmetic. The cosmetic may be a substance for topical application intended to alter a person's appearance, e.g., a liquid foundation, a powder foundation, blush, or lipstick. The cosmetic may be any substance recited in the Food and Drug Administration regulations, e.g., under 21 C.F.R. § 720.4.

The preparation, e.g., cosmetic may be at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, the formulations, compositions, or preparations described herein, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

Other components may be added to pharmaceutical formulations, e.g., preparations, or cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

In some embodiments, the preparation may be disposed in, or provided as, a powder, cosmetic, cream, stick, aerosol, salve, wipe, or bandage.

In some embodiments, the composition is associated with a cream. The cream may be a fluid comprising a thickening agent, and generally has a consistency that allows it to be spread evenly on the skin. Exemplary creams include moisturizing lotion, face cream, and body lotion.

In some embodiments, the composition is associated with a stick. A stick is typically a solid that, when placed in contact with a surface, transfers some of the stick contents to the surface. Exemplary sticks include deodorant stick, lipstick, lip balm in stick form, and sunscreen applicator sticks.

In some embodiments, the composition is associated with an aerosol. An aerosol is typically a colloid of fine solid particles or fine liquid droplets, in a gas such as air. Aerosols may be created by placing the ammonia oxidizing bacteria and/or nitro-hydrocarbon/nitro-fatty acid (and optionally carriers) in a vessel under pressure, and then opening a valve to release the contents. The container may be designed to only exert levels of pressure that are compatible with ammonia oxidizing bacteria viability. For instance, the high pressure may be exerted for only a short time, and/or the pressure may be low enough not to impair viability. Examples of consumer uses of aerosols include for sunscreen, deodorant, perfume, hairspray, and insect repellant.

In some embodiments, the composition is associated with a salve. A salve may be a topically applied agent with a liquid or cream-like consistency, intended to protect the skin or promote healing. Examples of salves include burn ointments and skin moisturizers.

In some embodiments, the composition is associated with a wipe. A wipe may be a flexible material suitable for topically applying a liquid or cream onto skin. The wipe may be, e.g., paper-based or cloth based. Exemplary wipes include tissues and wet wipes.

The compositions may also comprise one or more of a moisturizing agent, deodorizing agent, scent, colorant, insect repellant, cleansing agent, or UV-blocking agent.

For instance, the moisturizing agent may be an agent that reduces or prevents skin dryness. Exemplary moisturizing agents include humectants (e.g., urea, glycerin, alpha hydroxy acids and dimethicone) and emollients (e.g., lanolin, mineral oil and petrolatum). Moisturizing agents may be included, e.g., in compositions containing creams, balms, lotions, or sunscreen.

A deodorizing agent may be an agent that reduces unwanted odors. A deodorizing agent may work by directly neutralizing odors, preventing perspiration, or preventing the growth of odor-producing bacteria. Exemplary deodorizing agents include aluminum salts (e.g., aluminum chloride or aluminum chlorohydrate), cyclomethicone, talc, baking soda, essential oils, mineral salts, hops, and witch hazel. Deodorizing agents are typically present in spray or stick deodorants, and can also be found in some soaps and clothing.

An insect repellant may be an agent that can be applied to surfaces (e.g., skin) that discourage insects and other arthropods from lighting on the surface. Insect repellants include DEET (N,N-diethyl-m-toluamide), p-menthane-3,8-diol (PMD), icaridin, nepetalactone, citronella oil, neem oil, bog myrtle, dimethyl carbate, Tricyclodecenyl allyl ether, and IR3535 (3-[N-Butyl-N-acetyl]-aminopropionic acid, ethyl ester).

A cleansing agent may be an agent that removes dirt or unwanted bacteria from a surface like skin. Exemplary cleansing agents include bar soaps, liquid soaps, and shampoos.

A UV-blocking agent may be an agent that can be applied to a surface to reduce the amount of ultraviolet light the surface receives. A UV-blocking agent may block UV-A and/or UV-B rays. A UV blocking agent can function by absorbing, reflecting, or scattering UV. Exemplary UV-blocking agents include absorbers, e.g., homosalate, octisalate (also called octyl salicylate), octinoxate (also called octyl methoxycinnamate or OMC), octocrylene, oxybenzone, and avobenzone, and reflectors (e.g., titanium dioxide and zinc oxide). UV-blocking agents are typically present in sunscreens, and can also be found in skin creams and some cosmetics.

In some embodiments, the composition is associated with a conditioner. Conditioner generally refers to a substance with cream-like consistency that can be applied to hair to improve its appearance, strength, or manageability.

In some embodiments, the composition is associated with a product intended to contact the hair, for example, a brush, comb, shampoo, conditioner, headband, hair elastic, hair nets, shower caps, hats, and hairpieces. Nitric oxide formed on the hair, away from the skin surface, may be captured in a hat, scarf or face mask and directed into inhaled air.

In some embodiments, the product comprising composition described herein is packaged. The packaging may serve to compact the product or protect it from damage, dirt, or degradation. The packaging may comprise, e.g., plastic, paper, cardboard, or wood. In some embodiments the packaging is impermeable to bacteria. In some embodiments the packaging is permeable to oxygen and/or carbon dioxide.

Methods of Treatment with Ammonia Oxidizing Bacteria and/or Nitro-Fatty Acids or Nitro-Hydrocarbons

The present disclosure provides various methods of treating diseases and conditions using compositions described herein, e.g., compositions comprising ammonia oxidizing bacteria and/or nitro-fatty acids or nitro-hydrocarbons. In embodiments, the methods comprise administering a composition described herein to a subject. In embodiments, the methods comprise administering ammonia oxidizing bacteria, e.g., a preparation of ammonia oxidizing bacteria, e.g., a natural product or a fortified natural product, or compositions, preparations, or formulations comprising a natural product or a fortified natural product. In embodiments, the methods comprise administering a composition comprising an AOB and/or a nitro-fatty acid or nitro-hydrocarbon to a subject.

The subject may be evaluated, e.g., evaluated for application of ammonia oxidizing bacteria, e.g., prior to administering the preparation of ammonia oxidizing bacteria to the subject. For example, the subject is selected based on the need for an anti-inflammatory agent, the risk for developing an inflammatory disorder, or a medical history including a previous inflammatory disorder. For example, a subject having a need for an anti-inflammatory agent, having a risk for developing an inflammatory disorder, or having a medical history of previous inflammatory disorder(s) is selected, e.g., prior to administering a composition described herein.

In embodiments, a subject is in need of an anti-inflammatory agent if he or she has a disorder related to inflammation, e.g., an inflammatory skin disorder. In embodiments, a subject is at risk for developing an inflammatory disorder if the subject has previously had an inflammatory disorder and e.g., does not currently exhibit symptoms of an inflammatory disorder. In embodiments, a subject is at risk for developing an inflammatory disorder if the subject has previously had or currently suffers from an autoimmune disorder. In embodiments, a subject is at risk for developing an inflammatory disorder if the subject has a family history of an inflammatory disorder, if the subject has had a viral or bacterial infection, e.g., human immunodeficiency virus (HIV), if the subject is obese, or if the subject smokes or has smoked.

In embodiments, the subject has an inflammatory disorder, e.g., an inflammatory skin disorder. In embodiments, the subject has a disorder related to inflammation. Exemplary inflammatory skin disorders include skin inflammation, acne, eczema, psoriasis, atopic dermatitis, contact dermatitis, allergic reaction, or rosacea. In embodiments, a disorder related to inflammation includes renal, cardiovascular, pulmonary, neurological, and endocrine disorders. In embodiments, disorders related to inflammation include ankylosing spondylitis, antiphospholipid antibody syndrome, gout, inflammatory arthritis, myositis, rheumatoid arthritis, scleroderma, Sjogren's syndrome, systemic lupus erythematosis, vasculitis, vascular disease, heart disease, atherosclerosis, angina, particularly chronic, stable angina pectoris, ischemic diseases, congestive heart failure, myocardial infarction, ischemia reperfusion injury, hypertension, allergies, autoimmune sensitization, renal disease (e.g., end stage renal disease, acute kidney failure, glomerulonephritis, membranoproliferative glomerulonephritis, interstitial nephritis, Berger's disease, pyelonephritis, lupus nephritis, Goodpasture's syndrome, Wegener's granulomatosis), Hypertension, primary Raynaud's phenomena (PRP), Systemic Sclerosis, primary Sjogren's syndrome, generalized fibrosis of the heart and kidneys, fibrotic hypertrophy, Diabetes type 2, type 1 diabetes, pruritus, sicca syndrome, osteoporosis, portal hypertension, neuropathy, and pancreatic insufficiency, Alzheimer's disease (AD), autoimmune disorders, Primary Biliary Cirrhosis, autoimmune hepatitis, fibromyaligia, chronic obstructive pulmonary disease, Congestive heart failure, Crohn's disease, or Amyotrophic lateral sclerosis. In some embodiments, a disorder related to inflammation is a disease associated with low nitrite levels.

The ammonia oxidizing bacteria that may be used to treat diseases and conditions include all the ammonia oxidizing bacteria compositions described in this application, e.g. a preparation of ammonia oxidizing bacteria, a natural product or a fortified natural product, or compositions, preparations, or formulations comprising a natural product or a fortified natural product.

In some embodiments, the compositions described herein are used to treat a subject. Subjects may include an animal, a mammal, a human, a non-human animal, a livestock animal, or a companion animal.

The present disclosure also provides a method of providing a nitro-hydrocarbon or nitro-fatty acid to a subject, comprising positioning an effective dose of ammonia oxidizing bacteria described herein in close proximity to the subject. Similarly, the present disclosure provides ammonia oxidizing bacteria as described herein for use in providing a nitro-hydrocarbon or nitro-fatty acid to a subject. Likewise, the present disclosure provides a use of ammonia oxidizing bacteria in the manufacture of a medicament or composition suitable for position in close proximity to a subject.

The present disclosure also provides a method of treating or preventing a skin disorder, e.g., skin inflammatory disorder, comprising topically administering to a subject in need thereof a therapeutically effective dose of ammonia oxidizing bacteria and/or nitro-fatty acids or nitro-hydrocarbons as described herein. Similarly, the present disclosure provides compositions comprising ammonia oxidizing bacteria and/or nitro-fatty acids or nitro-hydrocarbons as described herein for use in treating a skin disorder in a subject. Likewise, the present disclosure provides a use of compositions comprising ammonia oxidizing bacteria and/or nitro-fatty acids or nitro-hydrocarbons as described herein in the manufacture of a medicament for treating skin disorder. In embodiments, the skin disorder is acne, rosacea, eczema, psoriasis, or urticaria; the skin infection is impetigo.

While not wishing to be bound by theory, it is proposed that treatment of acne with a therapeutically effective dose of ammonia oxidizing bacteria is achieved through production by the AOB of nitro-fatty acids and nitro-hydrocarbons, which have anti-inflammatory properties.

While not wishing to be bound by theory, it is proposed that treatment of rosacea with a therapeutically effective dose of ammonia oxidizing bacteria as described herein is achieved through production by the AOB of nitro-fatty acids and nitro-hydrocarbons, which have anti-inflammatory properties.

While not wishing to be bound by theory, it is proposed that treatment of eczema and/or atopic dermatitis with a therapeutically effective dose of ammonia oxidizing bacteria as described herein is achieved through production by the AOB of nitro-fatty acids and nitro-hydrocarbons, which have anti-inflammatory properties.

While not wishing to be bound by theory, it is proposed that treatment of psoriasis with a therapeutically effective dose of ammonia oxidizing bacteria described herein is achieved through production by the AOB of nitro-fatty acids and nitro-hydrocarbons, which have anti-inflammatory properties.

While not wishing to be bound by theory, it is proposed that treatment of psoriasis with a therapeutically effective dose of ammonia oxidizing bacteria as described herein may involve downregulation of inflammation due to NO generation, and e.g., production of nitro-fatty acids and/or nitro-hydrocarbons which have anti-inflammatory properties.

In some embodiments, this disclosure provides methods of using ammonia oxidizing bacteria as described herein to prevent a disease or disorder, e.g., a skin disorder. Prevention, in certain embodiments, means reducing the risk of a subject developing a disease, compared to a similar untreated subject. The risk need not be reduced to zero.

One suitable method of topical application is to apply sufficient ammonia oxidizing bacteria and then wear sufficient clothing so as to induce sweating. However, many people will want to derive the benefits of ammonia oxidizing bacteria while maintaining their current bathing habits, in which case, a culture of the bacteria can be applied along with sufficient substrate for them to produce NO. A nutrient solution approximating the inorganic composition of human sweat can be used for this purpose. Using bacteria adapted to media approximating human sweat minimizes the time for them to adapt when applied. Since sweat evaporates once excreted onto the skin surface, using a culture media that has a higher ionic strength is desirable. A concentration approximately twice that of human sweat is suitable, but other conditions are also contemplated. Ammonia oxidizing bacteria's nutritional needs are typically met with NH₃ or urea, O₂, CO₂, and minerals. In some embodiments, the substrate comprises trace minerals including iron, copper, zinc, cobalt, molybdenum, manganese, sodium, potassium, calcium, magnesium, chloride, phosphate, sulfate, or any combination thereof.

In some embodiments, the present disclosure provides a method of treating a skin disorder by applying a bandage comprising ammonia oxidizing bacteria to the site of the affected skin. Also provided are methods of producing such a bandage. The bandage may comprise, for example, an adhesive portion to affix the bandage to undamaged skin near the wound and a soft, flexible portion to cover or overlay the wound. In some embodiments, the bandage contains no other organisms but ammonia oxidizing bacteria. The bandage may made of a permeable material that allows gasses like oxygen and carbon dioxide to reach the ammonia oxidizing bacteria when the bandage is applied to the wound. In certain embodiments, the bandage comprises nutrients for ammonia oxidizing bacteria such as ammonium, ammonia, urea, or trace minerals. In certain embodiments, the bandage comprises an antibiotic to which the ammonia oxidizing bacteria is resistant. The antibiotic resistance may arise from one or more endogenous resistance gene or from one or more transgenes. In some embodiments, the bandage further comprises a nitro-fatty acid and/or nitro-hydrocarbon.

In some embodiments, the ammonia oxidizing bacteria e.g., a preparation of ammonia oxidizing bacteria, is administered at a dose of about 10⁸-10⁹ CFU, 10⁹-10¹⁰ CFU, 10¹⁰-10¹¹ CFU, 10¹¹-10¹² CFU, 10¹²-10¹³ CFU, or 10¹³-10¹⁴ CFU per application or per day. In some embodiments, the ammonia oxidizing bacteria is administered topically at a dose of about 10⁹-10¹⁰ CFU, about 1×10⁹-5×10⁹, 1×10⁹-3×10⁹, or 1×10⁹-10×10⁹ CFU; or about 10¹⁰-10¹¹ CFU, e.g., about 1×10¹⁰-5×10¹⁰, 1×10¹⁰-3×10¹⁰, or 1×10¹⁰-2×10¹⁰ CFU; or about 10¹¹-10¹² CFU, e.g., about 1×10¹¹-5×10¹¹, 1×10¹¹-3×10¹¹, or 1×10¹¹-2×10¹¹ CFU; or about 10¹² 10¹³ CFU, e.g., about 1×10¹²-5×10¹², 1×10¹²-3×10¹², or 1×10¹²-2×10¹² CFU; or about 10¹³-10¹⁴ CFU, e.g., about 1×10¹³-5×10¹³, 1×10¹³-3×10¹³, or 1×10¹³-2×10¹³ CFU.

In some embodiments, the ammonia oxidizing bacteria is administered in a volume of about 1-2, 2-5, 5-10, 10-15, 12-18, 15-20, 20-25, or 25-50 ml per dose. In some embodiments, the solution is at a concentration of about 10⁸-10⁹, 10⁹-10¹⁰, or 10¹⁰-10¹¹ CFUs/ml. In some embodiments, the ammonia oxidizing bacteria is administered as two 15 ml doses per day, where each dose is at a concentration of 10⁹ CFU/ml.

In embodiments, the composition described herein, e.g., comprising ammonia oxidizing bacteria, is administered topically, and in some cases, delivers a dose of 3×10⁶ to 3×10⁷ AOB cells/cm² of skin.

In some embodiments, the composition described herein is administered once, twice, three, or four times per day. In some embodiments, the composition described herein is administered once, twice, three, four, five, or six times per week. In some embodiments, the composition described herein is administered shortly after bathing. In some embodiments, the composition described herein is administered shortly before sleep.

In some embodiments, the composition described herein is administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the composition described herein is administered for an indefinite period of time, e.g., greater than one year, greater than 5 years, greater than 10 years, greater than 15 years, greater than 30 years, greater than 50 years, greater than 75 years.

In certain aspects, the present disclosure provides combination therapies comprising ammonia oxidizing bacteria and a second therapeutic. For instance, the disclosure provides physical admixtures of the two (or more) therapies are physically admixed. In other embodiments, the two (or more) therapies are administered in combination as separate formulation. The second therapy may be, e.g., a pharmaceutical agent, surgery, or any other medical approach that treats the relevant disease or disorder. For example, the second therapy comprises an anti-inflammatory agent.

Exemplary anti-inflammatory agents include but are not limited to nonsteroidal anti-inflammatory drug (i.e., an NSAID), (e.g., propionic acid derivatives, such as ibuprofen, naproxen, naproxen sodium, fenoprofen, ketoprofen, flurbiprofen and oxaprozin; sulindac; salicylic acid derivatives, such as salicylic acid, acetylsalicylic acid (aspirin), methyl salicylate, diflunisal, salsalate, olsalazine and sulfasalazine; para-aminopheriol derivatives, such as acetaminophen; indole and indene acetic acids, such as indomethacin, sulindac and etodolac; fenamates, such as mefenamic, meclofenamic, flufenamic, tolfenamic and etofenamic; heteroaryl acetic acids, such as tolmetin, ketorolac and diclofenac; enolic acids, such as the oxicam derivatives piroxicam, meloxicam, ampiroxicam, droxicam, pivoxicam, lornoxicam, cinnoxicam, sudoxicam and tenoxicam, and the pyrazolon derivatives phenylbutazone, oxyphenbutazone, antipyrine, aminopyrine and dipyrone; alkanones, such nambumetone; apazone; and nimesulide); and corticosteroids (e.g., hydrocortisone, flurandrenolide).

In a combination therapy capable of treating acne, the second therapy may comprise, e.g., a medication (e.g., systemic or topical) such as Benzoyl peroxide, antibiotics (such as erythromycin, clindamycin, or a tetracycline), Salicylic acid, hormones (e.g., comprising a progestin such as desogestrel, norgestimate or drospirenone), retinoids such as tretinoin, adapalene, tazarotene, or isotretinoin. The second therapy may also be a procedure such as comedo extraction, corticosteroid injection, or surgical lancing. The combination therapy may comprise one or more of the above-mentioned treatments.

In a combination therapy capable of treating rosacea, the second therapy may comprise, e.g., an antibiotic, e.g., an oral tetracycline antibiotic such as tetracycline, doxycycline, or minocycline, or a topical antibiotic such as metronidazole; azelaic acid; alpha-hydroxy acid; isotretinoin can be prescribed; sandalwood oil; clonidine; beta-blockers such as nadolol and propranolol; antihistamines (such as loratadine); mirtazapine; methylsulfonylmethane or silymarin, optionally in combination with each other; lasers such as dermatological vascular laser or CO₂ laser; or light therapies such as intense pulsed light, low-level light therapy or photorejuvenation. The combination therapy may comprise one or more of the above-mentioned treatments.

In a combination therapy capable of treating eczema, the second therapy may comprise, e.g., a corticosteroid such as hydrocortisone or clobetasol propionate, immunosuppressants (topical or systemic) such as pimecrolimus, tacrolimus, ciclosporin, azathioprine or methotrexate, or light therapy such as with ultraviolet light. The combination therapy may comprise one or more of the above-mentioned treatments.

In a combination therapy capable of treating psoriasis, the second therapy may comprise, e.g., a corticosteroid such as desoximetasone; a retinoid; coal tar; Vitamin D or an analogue thereof such as paricalcitol or calcipotriol; moisturizers and emollients such as mineral oil, vaseline, calcipotriol, decubal, or coconut oil; dithranol; or fluocinonide. The combination therapy may comprise one or more of the above-mentioned treatments.

In embodiments, the second therapy, e.g., anti-inflammatory agent, is administered concurrently with, before (e.g., at least 1 day, 2 days, 3 days, 4 days, 5 days, 6 days, 7 days, 1 week, 2 weeks, 4 weeks, 1 month, 2 months, 4 months, 6 months or more before), or after (e.g., at least 1 day, 2 days, 3 days, 4 days, 5 days, 6 days, 7 days, 1 week, 2 weeks, 4 weeks, 1 month, 2 months, 4 months, 6 months or more after) administration of the composition comprising AOB.

In embodiments, the second therapy, e.g., anti-inflammatory agent, is formulated together with the compositions comprising AOB or formulated separately from the composition comprising AOB.

In embodiments, the subject has previously been treated with the second therapy, e.g., an anti-inflammatory agent described herein, and for example, the subject exhibited a partial response or no response to the therapy. In embodiments, administration of a composition comprising AOB described herein in combination with the second therapy increases the efficacy of the second therapy compared to administration of the second therapy alone. In embodiments, administration of the composition comprising AOB in combination with the second therapy increases the efficacy of the AOB-comprising composition compared to administration of the AOB-comprising composition alone. In other embodiments, administration of a combination of a composition comprising AOB with a second therapy described herein permits the administration of either therapy at a lower dosage than is normally required for efficacy (e.g., for a reduction in a symptom of the disorder, e.g., inflammation related disorder). In embodiments, a dosage of AOB normally required for efficacy is described herein. In embodiments, a dosage of a second therapy normally required for efficacy is the dosage range described in the prescribing information of the second therapy for a particular indication. Without being bound by theory, it is believed that combining AOB and/or nitro-fatty acids or nitro-hydrocarbons with an anti-inflammatory agent decreases the dose normally required for either therapy alone to achieve an anti-inflammatory effect in a subject.

In some embodiments, co-administration of an anti-inflammatory agent with a composition comprising AOB described herein is not required to achieve an anti-inflammatory effect. Accordingly, in embodiments, a methods described herein does not comprise administering an anti-inflammatory agent concurrently with or within 6 months of administration of a composition comprising AOB. In embodiments, a method described herein does not comprise administering a second agent concurrently with or within 6 months of administration of a composition comprising AOB.

### Methods of Manufacturing a Nitro-Fatty Acid or Nitro-Hydrocarbon

Provided herein are methods of manufacturing a nitro-fatty acid or a nitro-hydrocarbon by using an AOB, e.g., an AOB described herein. In embodiments, manufacturing a nitro-fatty acid or nitro-hydrocarbon by using an AOB overcomes some drawbacks of synthesizing nitro-fatty acids/nitro-hydrocarbons chemically, such as challenges with obtaining pure regioisomers and sensitivity to alkaline conditions. See, e.g., Woodcock et al. Nitrated fatty acids: synthesis and measurement. Free Radic. Biol. Med. 59 (2013):14-26.

In embodiments, AOB are cultured as described herein. In some cases, the methods of manufacture include contacting an AOB with a substrate, such as an unsaturated fatty acid or an unsaturated hydrocarbon. Exemplary unsaturated fatty acids include oleic acid, linoleic acid, arachidonic acid, sebaleic acid, and sapienic acid. An exemplary unsaturated hydrocarbon includes squalene. In some examples, the methods of manufacture include supplying an unsaturated fatty acid or unsaturated hydrocarbon to a culture of AOB.

In embodiments, an unsaturated fatty acid or unsaturated hydrocarbon is contacted with of AOB. In embodiments, an unsaturated fatty acid or unsaturated hydrocarbon is added to a culture of AOB at a concentration of 0.001 to 1 millimoles/L (e.g., 0.001-0.01, 0.005-0.05, 0.01-0.1, 0.05-0.5, or 0.1-1 millimoles/L) unsaturated fatty acid or unsaturated hydrocarbon. In embodiments, the unsaturated fatty acid or unsaturated hydrocarbon is added to the culture of AOB while the AOB is at a density of 10̂7 to 10̂12 AOB cells/mL (e.g., 10̂7 to 10̂8, 10̂8 to 10̂9, 10̂9 to 10̂10, 10̂10 to 10̂11, or 10̂11 to 10̂12 AOB cells/mL). In some examples, the mixture of AOB and unsaturated fatty acid/unsaturated hydrocarbon is incubated for 1 hour to 7 days (e.g., 1-2 h, 2-4 h, 4-6 h, 6-12 h, 12-24 h, 1-2 days, 2-3-days, 3-4 days, 4-5 days, 5-6 days, or 6-7 days). For example, the mixture of AOB and unsaturated fatty acid/unsaturated hydrocarbon is incubated at a temperature of 4° C. to 40° C. (e.g., 4° C. to 10° C., 10° C. to 20° C., 20° C. to 30° C., 30° C. to 35° C., or 35° C. to 40° C.).

In other embodiments, an unsaturated fatty acid or unsaturated hydrocarbon is incubated with an isolated, e.g., purified, AOB preparation, e.g., an AOB preparation described herein. For example, the unsaturated fatty acid or unsaturated hydrocarbon is incubated with the isolated or purified AOB preparation such that the mixture contains a concentration of 10̂7 to 10̂12 AOB cells/mL (e.g., 10̂7 to 10̂8, 10̂8 to 10̂9, 10̂9 to 10̂10 to 10̂11, or 10̂11 to 10̂12 AOB cells/mL) and a concentration of 0.001 to 1 millimoles/L (e.g., 0.001-0.01, 0.005-0.05, 0.01-0.1, 0.05-0.5, or 0.1-1 millimoles/L) unsaturated fatty acid/unsaturated hydrocarbon. The mixture is incubated, e.g., for 1 hour to 7 days (e.g., 1-2 h, 2-4 h, 4-6 h, 6-12 h, 12-24 h, 1-2 days, 2-3-days, 3-4 days, 4-5 days, 5-6 days, or 6-7 days).

In embodiments, after incubation of an unsaturated fatty acid/unsaturated hydrocarbon with an AOB, a nitro-hydrocarbon or nitro-fatty acid is isolated, e.g., purified from the starting materials, to yield a resulting preparation of manufactured nitro-hydrocarbon or nitro-fatty acid. In embodiments, purification of a nitro-hydrocarbon or nitro-fatty acid from AOB cells comprises a filtration or centrifugation step. Filtration and centrifugation methods to remove cells are standard methods in the art. In embodiments, an unreacted unsaturated fatty acid or unreacted unsaturated hydrocarbon is separated from a nitro-fatty acid or nitro-hydrocarbon by methods such as solvent extraction, chromatography (e.g., column chromatography), and/or filtration. In embodiments, the separation of AOB cells occurs before the separation of unreacted unsaturated fatty acid or unreacted unsaturated hydrocarbon. In other embodiments, the separation of AOB cells occurs after the separation of unreacted unsaturated fatty acid or unreacted unsaturated hydrocarbon. In embodiments, the separation of AOB cells occurs concurrently with the separation of unreacted unsaturated fatty acid or unreacted unsaturated hydrocarbon.

In embodiments, the method does not comprise separating AOB from nitro-fatty acids or nitro-hydrocarbons. In embodiments, the method comprises killing AOB in the mixture, e.g., by heat, e.g., incubating the mixture at temperatures of at least 50° C., e.g., at least 50° C., 60° C., 70° C., 80° C., 90° C., or 100° C. In other embodiments, the method comprises killing AOB in the mixture, e.g., by sonication. Sonication methods are commonly known in the art.

In embodiments, the resulting preparation of manufactured nitro-fatty acid/nitro-hydrocarbon comprises less than 90%, e.g., less than 90%, 80%, 70%, 60%, 50%, 40%, 30%, 20%, 10%, 5%, 2.5%, or 1% of AOB by weight. In embodiments, the resulting preparation of manufactured nitro-fatty acid/nitro-hydrocarbon comprises less than 90%, e.g., less than 90%, 80%, 70%, 60%, 50%, 40%, 30%, 20%, 10%, 5%, 2.5%, or 1% of live AOB by weight. In embodiments, the resulting preparation of manufactured nitro-fatty acid/nitro-hydrocarbon comprises more than 10%, e.g., more than 10%, 20%, 30%, 40%, 50%, 60%, 70%, 80%, 90%, 95%, or 99% (by weight), of AOB, e.g., live AOB, or dead AOB. In embodiments, the resulting preparation of manufactured nitro-fatty acid/nitro-hydrocarbon comprises less than 90%, e.g., less than 90%, 80%, 70%, 60%, 50%, 40%, 30%, 20%, 10%, 5%, 2.5%, or 1% of unreacted unsaturated fatty acid or unsaturated hydrocarbon by weight.

In embodiments, the resulting preparation is incorporated into a cosmetic or pharmaceutical product. For example, the resulting preparation is incorporated into a cosmetic product described herein. In other examples, the resulting preparation is incorporated into a pharmaceutical product described herein.

The practice of the present disclosure may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F. M. Ausubel, et al. eds., current edition).

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

The function and advantages of these and other embodiments will be more fully understood from the following non-limiting example. The example is intended to be illustrative in nature and is not to be considered as limiting the scope of the embodiments discussed herein.

### Example

Conjugated linoleic acid (LA) was reacted with sodium nitrite. The reactions took place in glass vials with mixing at room temperature. The volume per reaction was 1.5 mL according to the following: (1 mL aqueous+/−1 mM NaNO₂+0.5 mL cLA in squalane). Table 1 (below) presents the four reactions that were evaluated.

TABLE 1
Reactions

| | Phosphate | Phosphate | | |
|---|---|---|---|---|
| | Buffer | Buffer | | |
| Reac- | (50 mM, | (50 mM, | Squalane + cLA | NaNO₂ |
| tion | pH 7.0) | pH 6.0) | (0.1 mM cLA) | (0.1M stock) |
| 1 | 1 mL | — | 0.5 mL | — |
| | | | | (control) |
| 2 | 1 mL | — | 0.5 mL | 10 μL |
| 3 | — | 1 mL | 0.5 mL | — |
| | | | | (control) |
| 4 | — | 1 mL | 0.5 mL | 10 μL |

The reactions at pH 7.0 were run as a control. The reactions at pH 6.0 were selected due to the proximity to the natural pH level of skin and because relatively more nitric oxide is generated from nitrite at pH 6.0 to drive the reaction. Reagent details are presented below in Table 2.

TABLE 2
Reagents

| | Manufac- | | Weight/ | | |
|---|---|---|---|---|---|
| Reagent | turer | Grade | Vol | | Cat. no. |
| Conjugated LA (cLA) | SIGMA | 99% | 5 | g | L1376-5G |
| Squalane | SIGMA | 99% | 100 | g | 234311- |
| | | | | | 100G |
| Sodium Nitrite, 0.1M | SIGMA | n/a | 1 | L | 70776-1L |
| Phosphate Buffer, | SIGMA | | 1 | L | 73173-1L |
| pH 7.0 (43 mM Na₂HPO₄ | | | | | |
| 2H₂O/28 mM KH₂PO₄) | | | | | |
| KH₂PO₄ Buffer conc., | SIGMA | n/a | 961.5 | mL | 01476- |
| pH 6.0 (50 mM KH₂PO₄/ | | | | | 961.5 mL |
| 5.6 mM Na from NaOH) | | | | | |

In terms of the preparation of the cLA stock and its subsequent dilution, 1 mM of cLA was prepared in 10 mL squalane (2.8 mg cLA in 10 mL squalane). The preparation was then diluted 10-fold in squalane to a 0.1 mM final concentration. 0.5 mL of the diluted preparation was then used for each reaction.

Samples were collected from both the aqueous and lipid phase at two and six hour sampling points. Full liquid chromatography-mass spectrometry (LC-MS) scans were then conducted for each reaction. Nitro-linoleic acid was detected at pH 6.0 at the six hour time point as evidenced by the LC-MS results. No nitro-linoleic acid was detected at pH 7.

Certain embodiments are within the following claims.

1. A composition comprising ammonia oxidizing bacteria (AOB) and a nitro-hydrocarbon or a nitro-fatty acid.

2. The composition of claim 1, wherein the nitro-fatty acid or the nitro-hydrocarbon is present at a concentration of 0.0005 to 2% by weight, e.g., 0.001 to 1% by weight (e.g., 0.001 to 0.01%, 0.005 to 0.05%, 0.01 to 0.1%, 0.05 to 0.5%, 0.1 to 1%, or 0.5% to 1% by weight).

3. The composition of claim 1 or 2, wherein the composition comprises a first nitro-fatty acid or nitro-hydrocarbon and a second nitro-fatty acid or nitro-hydrocarbon, and wherein the first nitro-fatty acid or nitro-hydrocarbon is present at a concentration higher than that of the second nitro-fatty acid or nitro-hydrocarbon, optionally wherein the first nitro-fatty acid or nitro-hydrocarbon is present at a concentration at least 1.5-fold (e.g., at least 1.5-fold, 3-fold, 5-fold, 10-fold, 20-fold, 40-fold, 80-fold, 100-fold, 200-fold, 1000-fold, or higher) higher than the concentration of the second nitro-fatty acid or nitro-hydrocarbon.

4. The composition of any of claims 1-3, wherein the AOB comprise live AOB, dead AOB, or a mixture of live and dead AOB.

5. The composition of claim 4, wherein 90% or fewer, e.g., 90%, 80%, 70%, 60%, 50%, 40%, 30%, 20%, 10%, 5%, 2.5%, 1%, or fewer, of the AOB are live.

6. The composition of claim 4, wherein 90% or fewer, e.g., 90%, 80%, 70%, 60%, 50%, 40%, 30%, 20%, 10%, 5%, 2.5%, 1%, or fewer, of the AOB are dead.

7. The composition of any of claims 1-6, wherein the composition comprises 90% of less, e.g., 90%, 80%, 70%, 60%, 50%, 40%, 30%, 20%, 10%, 5%, 2.5%, 1%, or less, by weight of AOB.

8. The composition of any of the previous claims, wherein the nitro-fatty acid a nitroalkene fatty acid derivative or a nitrohydroxy fatty acid derivative.

9. The composition of claim 8, wherein the nitro-fatty acid comprises nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

10. The composition of any of the previous claims, wherein the nitro-hydrocarbon comprises nitro-squalene.

11. The composition of any of the previous claims, wherein the composition is suitable for cosmetic use or pharmaceutical use.

12. A method of treating or preventing a disorder in a subject comprising topically administering to the subject a composition comprising AOB.

13. The method of claim 12, comprising topically administering to the subject the composition of any of claims 1-11.

14. The method of claim 12 or 13, comprising a step of selecting the subject based on (i) the need for an anti-inflammatory agent, (ii) the risk for developing an inflammatory disorder, or (iii) a medical history comprising a previous inflammatory disorder.

15. The method of claim 12 or 13, wherein the subject (i) is in need of an anti-inflammatory agent, (ii) is at risk for developing an inflammatory disorder, or (iii) has previously had an inflammatory disorder.

16. The method of claim 15, wherein the subject has previously been administered an anti-inflammatory agent, e.g., an anti-inflammatory agent other than a composition comprising AOB.

17. The method of claim 15, wherein the subject exhibited a partial response or no response to the previously administered anti-inflammatory agent.

18. The method of any of claims 12-17, further comprising administering to the subject an anti-inflammatory agent in combination with the composition comprising AOB.

19. The method of claim 18, wherein the anti-inflammatory agent is administered concurrently with, before, or after administration of the composition comprising AOB.

20. The method of claim 18, wherein the anti-inflammatory agent and the composition comprising AOB are formulated together or separately.

21. The method of any of claims 18-20, comprising administering a dose of the anti-inflammatory agent that is lower than the average recommended dose.

22. The method of any of claims 12-15, wherein the method does not comprise administering an anti-inflammatory agent concurrently with, less than 6 months before, or less than 6 months after administration of the composition comprising AOB.

23. The method of any of claims 12-22, wherein the composition comprising AOB is administered at a dose of 3×10⁶ to 3×10⁷ AOB cells/cm² of skin.

24. The method of any of claims 12-23, wherein the composition comprising AOB is administered every 1, 2, 3, 4, 5, 6, or 7 days, or every 1, 2, 3, 4, 5, 6, 7, 8, 9, or 10 weeks, or every month.

25. The method of any of claims 12-24, wherein the composition comprising AOB is administered for at least 1 week, e.g., at least 1, 2, 3, 4, 5, 6, 7, 8, 9, 10 weeks, or at least 1 month, e.g., at least 1, 2, 3, 4, 5, 6, 7, 8, 9, 10, 11, 12 months, or at least 1 year, e.g., at least 1, 2, 3, 4, 5, 6, 7, 8, 9, 10, or 15 years.

26. The method of any of claims 12-25, wherein the composition comprising AOB is formulated with an excipient, e.g., an excipient described herein.

27. The method of any of claims 12-26, wherein the composition comprising AOB further comprises a nitro-fatty acid or a nitro-hydrocarbon.

28. The method of any of claims 12-27, wherein the disorder comprises a skin disorder, e.g., skin inflammation, acne, eczema, psoriasis, or rosacea.

29. The method of any of claims 12-27, wherein the disorder is a renal, cardiovascular, pulmonary, neurological, or endocrine disorder.

30. The method of any of claims 12-29, wherein the composition comprises an activated AOB, e.g., AOB in a growth state.

31. The method of any of claims 12-29, wherein the composition comprises an AOB in an inactive state, e.g., a storage state.

32. The method of claim 31, wherein the composition comprises a first compartment comprising the AOB in a storage state and a second compartment comprising an activator, e.g., ammonia or oxygen, wherein the first compartment and the second compartment are physically separated by a barrier.

33. The method of claim 32, wherein the method comprises disrupting the barrier between the first compartment and the second compartment, wherein upon disruption of the barrier, the AOB and the activator mix or contact one another, thereby producing activated AOB.

34. The method of claim 33, comprising depositing the composition comprising AOB on a skin surface of the subject prior to disruption of the barrier.

35. The method of claim 33, comprising depositing the composition comprising AOB on a skin surface of the subject after disruption of the barrier.

36. A method of manufacturing a nitro-fatty acid or nitro-hydrocarbon comprising incubating an AOB with an unsaturated fatty acid or unsaturated hydrocarbon, and optionally separating the nitro-fatty acid or nitro-hydrocarbon from unreacted fatty acid or unreacted hydrocarbon and/or from AOB, thereby manufacturing a purified NOFA preparation.

37. The method of claim 36, wherein the separating step comprises filtration, centrifugation, chromatography (e.g., column chromatography), and/or solvent extraction.

38. The method of any of claims 36-37, wherein the purified nitro-fatty acid or nitro-hydrocarbon preparation comprises 50% or less, e.g., 50%, 40%, 30%, 20%, 10%, 5%, 2.5%, 1% or less, by weight of AOB.

39. The method of any of claims 36-38, wherein the purified nitro-fatty acid or nitro-hydrocarbon preparation comprises 50% or less, e.g., 50%, 40%, 30%, 20%, 10%, 5%, 2.5%, 1%, 0.5%, 0.1%, 0.05%, 0.01%, 0.005%, 0.001%, 0.0005%, 0.0001%, or less, by weight of the un-nitrated unsaturated fatty acid or un-nitrated unsaturated hydrocarbon.

40. The method of any of claims 36-39, wherein the purified nitro-fatty acid or nitro-hydrocarbon preparation comprises 0.0001% or more, e.g., 0.0001%, 0.0005%, 0.001%, 0.005%, 0.01%, 0.05%, 0.1%, 0.5%, 1%, 5%, 10%, 20%, 30%, 40%, 50%, 60%, 70%, 80%, 90%, 95%, 99% or more of the nitro-fatty acid or nitro-hydrocarbon.

41. The method of any of claims 36-40, wherein the purified nitro-fatty acid or nitro-hydrocarbon preparation comprises a nitroalkene fatty acid derivative or a nitrohydroxy fatty acid derivative.

42. The composition of claim 41, wherein the purified nitro-fatty acid or nitro-hydrocarbon preparation comprises nitro-oleic acid (e.g., 9-nitro-octadecenoid acid or 10-nitro-octadecenoic acid), nitro-conjugated linoleic acid (e.g., 9-cis,11-trans-octadecadienoic acid), nitroarachidonic acid, a nitrohydroxy derivative of oleate, a nitrohydroxy derivative of linoleate, a nitrohydroxy derivative of linolenate, a nitro derivative of sebaleic acid, a nitrohydroxy derivative of sebaleic acid, a nitro derivative of sapienic acid, or a nitrohydroxy derivative of sapienic acid.

43. A method of identifying an active strain of AOB, comprising detecting the level of nitro-fatty acid or nitro-hydrocarbon produced by a candidate AOB strain, wherein production of a level of nitro-fatty acid or nitro-hydrocarbon above a control level indicates that the strain is active.
